(19) United States
(12) Patent Application Publication
(10) Pub. No.: US 2026/0325720 A1
(43) Pub. Date: Oct. 22, 2026

(54) NANOPARTICLE-FUNCTIONALIZED ELECTROSPUN SCAFFOLDS AND USES THEREOF

(71) Applicant: The Trustees of Columbia University in the City of New York, New York, NY (US)

(72) Inventors: Santiago Correa, New York, NY (US); Treena L. Arinzeh, West Orange, NJ (US); Jose McFaline-Figueroa, New York, NY (US)

(21) Appl. No.: 19/468,525

(22) Filed: Feb. 3, 2026

Related U.S. Application Data
(60) Provisional application No. 63/754,713, filed on Feb. 6, 2025.
(60) Provisional application No. 63/902,952, filed on Oct. 21, 2025.

(51) Int. Cl. A61L 27/54; A61L 27/16; A61L 27/28; A61L 27/38; C12N 5/00; C12N 5/078

(52) U.S. Cl. CPC A61L 27/54; A61L 27/16; A61L 27/28; A61L 27/38; C12N 5/0062; C12N 5/0634; A61L 2300/252; C12N 2513/00; C12N 2533/30

(57) ABSTRACT

Compositions and methods of making of piezoelectric polymer scaffolds and a plurality of charged nanoparticles and methods of use thereof.

### CROSS-REFERENCE TO RELATED APPLICATIONS

The present application claims priority to and the benefit of U.S. Provisional Patent Application Ser. No. 63/902,952, filed Oct. 21, 2025, and entitled “NANOPARTICLE FUNCTIONALIZED ELECTROSPUN SCAFFOLDS AND USES THEREOF” and U.S. Provisional Patent Application Ser. No. 63/754,713, filed Feb. 6, 2025, and entitled “NANOPARTICLE FUNCTIONALIZED ELECTROSPUN SCAFFOLDS AND USES THEREOF,” the entire contents of both of which are hereby incorporated by reference herein.

### FIELD OF THE DISCLOSURE

The present disclosure relates generally relates to nanoparticle-functionalized electrospun scaffolds and uses thereof.

### BACKGROUND OF THE INVENTION

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

Advancements in nanotechnology have influenced therapeutic strategies, particularly in tissue engineering and regenerative medicine. Nanoparticles, due to their adjustable size, surface modification capabilities, and biosystem compatibility, can offer a unique and versatile platform in these fields.

Nanoparticles that are integrated into electrospun scaffolds can be used in mitigating inflammatory responses and providing a conducive environment for neuronal survival and regeneration. These nanoparticle-enhanced scaffolds can effectively harness biological cues to prolong the stability and bioactivity within the central nervous system (CNS).

These cues include specific cytokines and growth factors crucial for modulating the inflammatory responses. In the context of CNS lesions, they can push astrocytes towards an anti-inflammatory state. However, the efficacy of these cues can be hampered by challenges in ensuring their stability, controlled delivery, and in vivo bioactivity.

Instructive biomaterials that modulate immune cell function are needed across regenerative medicine, cancer immunotherapy, and ex-vivo cell engineering. Local delivery from a material interface can reduce systemic immune-related adverse effects,¹⁻⁶, yet presenting potent immune signals such as cytokines in a sustained, spatially confined manner remains difficult. Cytokines are rapidly degraded in vivo⁷⁻⁹, and often act most effectively when recognized in a membrane-proximal configuration,^{9,10} which complicates controlled use in biomaterials. These features complicate their controlled use in biomaterial systems. As a result, there is a need for tunable biomaterials to locally present cytokines at the cell interface.

Prior strategies to localize bioactive cues within biomaterials include bulk encapsulation,¹¹ covalent tethering,¹² and immobilization onto nanoparticles,¹³ each with tradeoffs in spatial precision, loading efficiency, and functional potency. In soluble form, cytokines typically require repeated dosing or chemical modification to extend half-life, with associated losses in activity.^{14,15} Liposomes offer a promising alternative because they can carry fragile proteins,^{16,17} limit systemic exposure,¹⁸ and mimic aspects of membrane-bound ligand display.^{19,20} However, methods to integrate liposomes uniformly and stably onto scaffolds, while preserving cell access to surface-presented cargo, remain limited.²¹⁻²⁵

Electrospun scaffolds are widely used in immunoengineering and tissue regeneration due to their high surface area,²⁶ tunable architecture,²⁷⁻²⁹ and mechanical responsiveness.³⁰⁻³³ These features make electrospun scaffolds attractive for presenting bioactive signals at the cell interface, yet reliably achieving such presentation remains challenging.

### BRIEF SUMMARY OF THE INVENTION

This work demonstrates in some embodiments, a surface-functionalized scaffold system based on electrospun PVDF-TrFE fibers, designed for tunable electrostatic adsorption of charged nanoparticles and liposomes. It was shown in some embodiments that mechanically generated surface charge during loading, together with optimized buffer conditions for pH and ionic strength, enables robust adsorption of both polymeric nanoparticles and soft lipid nanocarriers without covalent modification. In some embodiments, the coated scaffolds remained cytocompatible and supported three-dimensional cell culture of HEK and Jurkat T cells. Building on a supramolecular tethering strategy previously applied in hydrogel systems,⁴⁰ interleukin-15 was noncovalently displayed on scaffold-bound liposomes and demonstrate contact-dependent receptor signaling in a reporter cell line. This work establishes a modular, bioactive interface localized cytokine presentation with potential applications in in situ immune modulation and ex-vivo T cell expansion.

In some embodiments, the techniques described herein relate to a composition including: (a) a piezoelectric polymer scaffold; and (b) a plurality of charged nanoparticles, non-covalently adsorbed onto the piezoelectric polymer scaffold.

In some embodiments, the techniques described herein relate to a method of improving three dimensionality of a cell culture including: (a) providing a composition including: (i) a piezoelectric polymer scaffold; and (ii) a plurality of charged nanoparticles, non-covalently adsorbed onto the piezoelectric polymer scaffold; (b) applying the composition into a cell culture environment, thereby improving the three dimensionality of the cell culture.

In some embodiments, the techniques described herein relate to a method of producing a composition including: (a) providing a piezoelectric polymer scaffold, wherein the piezoelectric polymer scaffold is electrospun; (b) providing a plurality of charged nanoparticles; (c) providing a buffer solution; (d) combining the piezoelectric polymer scaffold, the plurality of charged nanoparticles, and the buffer solution; (e) applying a vacuum to the buffer solution, and repeatedly agitating the buffer solution, so as to thereby non-covalently adsorb the plurality of charged nanoparticles onto the piezoelectric polymer scaffold and produce the composition.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1C depict systematic comparison of exemplary loading methodology on charged particles which reveals optimal loading technique;

FIGS. 2A-2B depict how solution conditions can tune nanoparticle loading onto scaffolds;

FIGS. 3A-3E depict electrostatic liposome adsorption onto PVDF-TrFE scaffolds;

FIGS. 4A-4D depict liposome-coated scaffolds are suitable substrates for cells and enable cytokine presentation;

FIG. 5 depicts nanoparticle summary data;

FIG. 6 depicts scaffold fiber size distribution;

FIG. 7 depicts anionic CML coating heterogeneity;

FIG. 8 depicts coefficients of variance between mean fluorescence intensity of coated scaffolds in different buffer conditions;

FIGS. 9A-9B depict nanoparticle dose response for scaffold coating;

FIG. 10 depicts uncropped SEM of polymeric nanoparticles on corona poled scaffolds (images from FIG. 2B), collected at 5-10K× magnification;

FIG. 11 depicts low-magnification SEM image of uncoated scaffold (left) compared to scaffold coated in 2 mg/mL LbL CML with HEPES-NaCl (right), images acquired at 1-1.5K×magnification;

FIG. 12 depicts piezoelectric coefficient decreases with all particle coatings;

FIG. 13 depicts cationic liposome coating heterogeneity;

FIG. 14 depicts liposome characterization after de-adsorption; and

FIG. 15 shows how particles are retained on scaffolds in aqueous environments.

### DETAILED DESCRIPTION OF THE INVENTION

The following is intended to be a description of the exemplary embodiments of the present disclosure and is not intended to limit the scope of the exemplary embodiments of the present disclosure.

### DEFINITIONS

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In some embodiments, about means within a standard deviation using measurements generally acceptable in the art. In some embodiments, about means a range extending to +/−10% of the specified value. In some embodiments, about includes the specified value. Unless otherwise indicated, the word “or” in the specification and claims is considered to be the inclusive “or” rather than the exclusive or, and indicates at least one of and any combination of items it conjoins. “And/or” as used herein, for example with option A and/or option B, encompasses the separate embodiments of (i) option A, (ii) option B, and (iii) option A plus option B.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

“And/or” as used herein, for example with option A and/or option B, encompasses the separate embodiments of (i) option A, (ii) option B, and (iii) option A plus option B.

### General

For the embodiments described herein, each embodiment disclosed herein is contemplated as being applicable to each of the other disclosed embodiments.

As used herein, all headings are simply for organization and are not intended to limit the disclosure in any manner. The content of any individual section may be equally applicable to all sections. All combinations of the various elements disclosed herein are within the scope of the invention.

### Overview of Compositions, Methods of Production, and Methods of Use

In some embodiments, compositions comprise piezoelectric scaffolds and a plurality of charged nanoparticles which are non-covalently adsorbed onto the piezoelectric scaffolds. In some embodiments, the plurality of charged particles comprise a biologically active molecule. In some embodiments, methods comprise the production of these compositions.

In some embodiments, the size of the compositions is on the scale of millimeters. In some embodiments, the size of the compositions is on the scale of centimeters. In some embodiments, the size of the compositions is on the scale of 0.1 meters to 1 meters. In some embodiments, the size of the compositions is on the scale of 0.1 cm to 1000 cm. In some embodiments, the lengths of the compositions are 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm. In some embodiments, the width of the compositions are 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm.

In some embodiments, methods comprise applying scaffolds and a plurality of charged nanoparticles which are non-covalently adsorbed onto the piezoelectric scaffolds for treating wounds, ex-vivo cell culture, and/or local administration of a drug for treating or reducing inflammation, stem cell-based and immune cell-based therapies.

### Piezoelectric Polymer Scaffold

A noncovalent surface-functionalization approach that preserves scaffold architecture and maintains nanocarrier bioactivity and cell accessibility is provided.

It may be that electrostatic self-assembly principles established in layer-by-layer systems are adapted to electrospun fibers to tether charged nanoparticles without covalent chemistry, with coating density tuned through surface charge and solution conditions.^{34,35} Piezoelectric polymers like poly(vinylidene fluoride-trifluoroethylene) (PVDF-TrFE) present a unique opportunity in this context: mechanical deformation can induce surface charge on the fibers, creating favorable conditions for adsorption of oppositely charged species.

In some embodiments, compositions and methods of production and use of the present invention comprise scaffolds. In some embodiments, the scaffolds are electrospun. In some embodiments, the scaffolds comprise piezoelectric scaffolds. In some embodiments, the scaffolds comprise polymer scaffolds. In some embodiments, the scaffolds comprise piezoelectric polymer scaffolds which are electrospun.

In some embodiments, the piezoelectric polymer scaffold is generated by solvent casting. In some embodiments, the piezoelectric polymer scaffold is generated by freeze casting. In some embodiments, the piezoelectric polymer scaffold is generated by 3D printing.

In some embodiments, the piezoelectric polymer scaffold comprises a piezoelectric fluoropolymer scaffold. In some embodiments, the piezoelectric polymer scaffold comprises poly(vinylidene fluoride-trifluoroethylene) (PVDF-TrFE). In some embodiments, the piezoelectric polymer scaffold comprises PVDF-hexafluoropropylene (PVD-HFP). In some embodiments, the piezoelectric polymer scaffold comprises poly(L-lactic acid) (PLLA) and/or polyhydroxyalkanoates (PHB/PHBV).

In some embodiments, the thickness of the scaffold fibers range from 0.5-100 μm. In some embodiments, the thickness of the scaffold fibers range from 1-10 μm. In some embodiments, the thickness of the scaffold fibers range from 1.5-2 μm.

In some embodiments, the piezoelectric polymer scaffold is corona poled.

### Nanoparticles

In some embodiments, compositions and methods of production and use comprise a plurality of charged nanoparticles.

### General Properties

In some embodiments, the plurality of charged nanoparticles are non-covalently adsorbed onto a scaffold. In some embodiments, the plurality of charged nanoparticles are non-covalently adsorbed onto the piezoelectric polymer scaffold. In some embodiments, non-covalent bonding comprises electrostatic adsorption. In some embodiments, the plurality of charged nanoparticles are electrostatically adsorbed onto the piezoelectric polymer scaffold

In some embodiments, the plurality of charged nanoparticles are cationic. In some embodiments, the plurality of charged nanoparticles are anionic. In some embodiments, the plurality of charged nanoparticles are a mix of anionic and cationic particles.

In some embodiments, the plurality of charged nanoparticles are negatively charged and are covalently adsorbed onto a positively charged piezoelectric polymer scaffold. In some embodiments, the plurality of charged nanoparticles are positively charged and are covalently adsorbed onto a negatively charged piezoelectric polymer scaffold.

In some embodiments, the plurality of charged nanoparticles comprise nanoparticles having diameters from 1-999 nm. In some embodiments, the plurality of charged nanoparticles comprise nanoparticles having diameters from 50-200 nm. In some embodiments, the plurality of charged nanoparticles comprise nanoparticles having diameters from 100-150 nm. In some embodiments, the diameter refers to a characteristic particle dimension, including an equivalent spherical diameter. In some embodiments, the diameter represents an average particle diameter, a median particle diameter, a mode, or another statistical measure of a particle size distribution.

In some embodiments, the diameter is determined using one or more conventional particle sizing techniques, including dynamic light scattering, electron microscopy, atomic force microscopy, laser diffraction, or combinations thereof, and may be reported as a number-weighted, volume-weighted, mass-weighted, or intensity-weighted value.

In some embodiments, the plurality of charged nanoparticles comprise charged nanoparticles having zeta potentials between −100 to +100 mV. In some embodiments, the plurality of charged nanoparticles comprise charged nanoparticles having zeta potentials between −50 and +50 mV. In some embodiments, the plurality of charged nanoparticles comprise charged nanoparticles having zeta potentials between −25 and +25 mV.

In some embodiments, the plurality of charged nanoparticles have a polydispersity index of less than 0.1, less than 0.05, or less than 0.03.

In some embodiments, the plurality of charged nanoparticles covers at least 10% of the total surface area of the piezoelectric polymer scaffold. In some embodiments, the plurality of charged nanoparticles covers at least 50% of the total surface area of the piezoelectric polymer scaffold. In some embodiments, the plurality of charged nanoparticles covers at least 75% of the total surface area piezoelectric polymer scaffold.

In some embodiments, the plurality of charged nanoparticles covers at least 10% of the surface area of a portion of the piezoelectric polymer scaffold. In some embodiments, the plurality of charged nanoparticles covers at least 50% of the surface area of a portion of the piezoelectric polymer scaffold. In some embodiments, the plurality of charged nanoparticles covers at least 75% of the surface area of a portion of the piezoelectric polymer scaffold

In some embodiments, the plurality of charged nanoparticles densely and/or uniformly coat the piezoelectric polymer scaffold. In some embodiments, the plurality of charged nanoparticles densely and/or uniformly coat the piezoelectric polymer scaffold as determined by microscopy. In some embodiments, the plurality of charged nanoparticles densely and/or uniformly coat the piezoelectric polymer scaffold as determined by confocal microscopy.

### Functionalized Polymers

In some embodiments, the plurality of charged nanoparticles comprise functionalized polymers. In some embodiments, the plurality of charged nanoparticles comprise functionalized polymer latex. In some embodiments, the plurality of charged nanoparticles comprise carboxylate modified latex.

In some embodiments, the plurality of charged nanoparticles comprise layer by layer nanoparticles. In some embodiments, the plurality of charged nanoparticles comprise layer by layer functionalized polymer nanoparticles. In some embodiments, the plurality of charged nanoparticles comprise layer by layer functionalized nanoparticles comprising one or more of cationic poly-allylamine hydrochloride (PAH), anionic poly(acrylic acid) (PAA), and anionic carboxylate modified latex.

In some embodiments, the polymers comprise co-polymers.

### Liposomes

In some embodiments, the plurality of charged nanoparticles comprises charged liposomes. In some embodiments, the liposomes comprise phospholipids, surfactants, and/or plant cholesterols. In some embodiments, the liposomes comprise phosphatidylcholine, di-chain surfactants, and/or cholesterols. In some embodiments, the liposomes comprise 1,2-distearoyl-sn-glycero-3-phosphocholine (DSPC), 1,2-dioleoyl-3-trimethylammonium-propane (DOTAP), and/or plant cholesterol. In some embodiments, the liposomes comprise 1,2-distearoyl-sn-glycero-3-phosphocholine (DSPC), 1,2-dioleoyl-3-trimethylammonium-propane (DOTAP), and plant cholesterol at a 9:1:2: molar ratio.

In some embodiments, the coating of the liposomes onto the piezoelectric polymer scaffold is measured by an increase in levels of elemental nitrogen in the piezoelectric polymer scaffold coated with the liposomes. In some embodiments, the increase in weight percent of elemental nitrogen is at least 0.83, 0.7, 0.6, or 0.5.

### Functionalization of Nanoparticles

In some embodiments, plurality of charged nanoparticles comprises functionalized charged particles. In some embodiments, the functionalized charged particles comprise an active agent. In some embodiments, the functionalized polymer latex nanoparticles, layer-by-layer coated nanoparticles, and/or liposome comprise an active agent. In some embodiments, the active agent comprises a plurality of active agents.

### Active Agents

In some embodiments, the active agent comprises a therapeutic agent. In some embodiments, the active agent is a biologically active molecule.

In some embodiments, the active agent comprises an anti-inflammatory agent, an antimicrobial agent, an anticancer agent, an analgesic or anesthetic agent, an antifibrotic agent, an angiogenic or anti-angiogenic agent, and/or a cardiovascular or metabolic modulator agent.

In some embodiments, the active agent comprises a drug. In some embodiments, the active agent comprises a small molecule. In some embodiments, the active agent comprises a steroid. In some embodiments, the active agent comprises a vitamin. In some embodiments, the active agent comprises an antibiotic. In some embodiments, the active agent comprises an antioxidant.

In some embodiments, the active agent comprises a biologic. In some embodiments, the active agent comprises a gene therapy nucleic acid. In some embodiments, the active agent comprises a cellular therapy. In some embodiments, the active agent comprises an immunotherapeutic agent.

In some embodiments, the active agent comprises an enzyme, antibody, an antibody fragment, a peptide, a protein, and/or an aptamer. In some embodiments, the active agent comprises a nucleic acid. In some embodiments, the nucleic acid comprises a DNA, RNA, siRNA, miRNA, mRNA, antisense oligonucleotide, and/or guide RNA.

In some embodiments, the active agent comprises a growth factor. In some embodiments, the growth factor comprises nerve growth factor. In some embodiments, the growth factor comprises epidermal growth factor. In some embodiments, the growth factor comprises insulin-like growth factor.

In some embodiments, the active agent comprises an immune modulator. In some embodiments, the active agent comprises a cytokine. In some embodiments, the active agent comprises an anti-inflammatory cytokine. In some embodiments, the active agent comprises interleukin-15 (IL-15). In some embodiments, the active agent comprises a chemokine.

In some embodiments, the active agent comprises a human sequence. In some embodiments, the active agent comprises a sequence that is identical to, or substantially identical to a human sequence. In some embodiments, the active agent has a non-immunogenic sequence for humans.

In some embodiments, the active agent comprises an adjuvant.

In some embodiments, the active agent comprises a functional biopolymer.

In some embodiments, the active agent generates a signal in response to the environment.

In some embodiments, the signal comprises an optical and/or chemical signal. In some embodiments, the signal is generated in response to one or more of chemical or physiochemical conditions, enzymatic activity, a molecular binding event, cellular activity, cellular presence, and/or exposure to a predefined compound.

### Presentation of Active Agents

In some embodiments, the functionalized charged particles encapsulate the active agent. In some embodiments, the bilayer of a liposome is selectively permeable and permits release of the active agent. In some embodiments, the functionalized charged particles release the active agent when a predetermined value is met for one or more of: pH, temperature, and/or exposure to radiation.

In some embodiments, the active agent is surface mounted to one or more of the functionalized charged particles. In some embodiments, the active agent is surface mounted to one or more of the functionalized charged particles via a linker. In some embodiments, the linker may be a polymer or carbohydrate. In some embodiments, the linker may be cleavable.

In some embodiments, the active agent is surface mounted to one or more of the functionalized charged particles via a histidine-metal interaction. In some embodiments, the active agent is surface mounted to one or more of the functionalized charged particles via a histidine-nickel interaction. In some embodiments, the active agent is surface mounted to one or more of the functionalized charged particles via a biological ligand-receptor interaction. In some embodiments, the active agent is surface mounted to one or more of the functionalized charged particles via an antibody or affinity protein capture.

In some embodiments, the active agent is bound to one or more of the functionalized charged particles via metal-chelating lipid. In some embodiments the metal-chelating lipid comprises nickel-chelating lipid DGS-NTA(Ni).

In some embodiments, the active agent comprises IL-15, and is optionally bound to a functionalized liposome via a nickel-chelating lipid DGS-NTA(Ni).

In some embodiments, the active agent remains active for up to 6 days. In some embodiments, the active agent remains active for up to 5 days. In some embodiments, the active agent remains active for up to 4 days. In some embodiments, the active agent remains active for up to 3 days. In some embodiments, the active agent remains active for up to 2 days. In some embodiments, the active agent remains active for up to 1 day.

In some embodiments, the functionalized charged particles provides localized delivery of an active agent. In some embodiments, the liposomes present active agents without releasing the active agents.

### Quantification of Coating

In some embodiments, the non-covalent adsorption of the plurality of charged nanoparticles onto the piezoelectric polymer scaffold is measured to quantify the adsorption.

In some embodiments, the non-covalent adsorption is measured via fluorescent imaging. In some embodiments, a plurality of charged nanoparticles is fluorescently tagged and adsorbed onto the piezoelectric polymer scaffold in a solution; the scaffold is washed to remove non-adsorbed fluorescently charged nanoparticles, and then imaged with microscopy to determine adsorption. In embodiments, the plurality of charged nanoparticles is inherently fluorescent and does not require tagging. In embodiments, the microscopy is confocal or widefield, to name a few examples.

In some embodiments, when the plurality of charged nanoparticles is fluorescently labeled, and the fluorescence is measured via an inverted confocal fluorescence microscope, the mean fluorescence intensity density per micron of the piezoelectric polymer scaffold is from 10 RFU/μm²-50 RFU/μm², 20 RFU/μm²-40 RFU/μm², or 30 RFU/μm²-35 RFU/μm².

### Plurality of Cells

In some embodiments, compositions of the present invention further comprise a plurality of cells.

In some embodiments, the plurality of cells comprises mammalian cells. In some embodiments, the plurality of cells comprises human cells.

In some embodiments, the plurality of cells comprises bacterial cells, fungal cells, insect cells, plant cells, or combinations thereof. In some embodiments, the plurality of cells comprises prokaryotic cells.

In some embodiments, the plurality of cells comprises tissue cells. In some embodiments, the plurality of cells comprises primary cells. In some embodiments, the plurality of cells comprises primary mammalian cells. In some embodiments, the plurality of cells comprises engineered mammalian cells. In some embodiments, the plurality of cells comprises immortalized cells.

In some embodiments, the plurality of cells comprises immune cells. In some embodiments, the plurality of cells comprises stromal cells. In some embodiments, the plurality of cells comprises epithelial cells. In some embodiments, the plurality of cells comprises endothelial cells. In some embodiments, the plurality of cells comprises mesenchymal cells. In some embodiments, the plurality of cells comprises parenchymal cells. In some embodiments, the plurality of cells comprises hematopoietic cells. In some embodiments, the plurality of cells comprises neurons. In some embodiments, the plurality of cells comprises glial cells. In some embodiments, the plurality of cells comprises vascular cells. In some embodiments, the plurality of cells comprises astrocytes.

In some embodiments, the plurality of cells comprises progenitor cells. In some embodiments, the plurality of cells comprises stem cells. In some embodiments, the plurality of cells comprises differentiated cells. In some embodiments, the plurality of cells comprises activated cells. In some embodiments, the plurality of cells comprises engineered cells. In some embodiments, the plurality of cells comprises co-cultured cell populations.

In some embodiments, the plurality of cells comprises an immortalized cell line.

In some embodiments, the plurality of cells comprises the HeLa cell line. In some embodiments, the plurality of cells comprises the CHO cell line. In some embodiments, the plurality of cells comprises the 3T3 cell line. In some embodiments, the plurality of cells comprises the COS-7 cell line. In some embodiments, the plurality of cells comprises the BHK-21 cell line. In some embodiments, the plurality of cells comprises the U2OS cell line. In some embodiments, the plurality of cells comprises the MDCK cell line.

In some embodiments, the plurality of cells comprises a HEK cell line. In some embodiments, the plurality of cells comprises the HEK293 cell line. In some embodiments, the plurality of cells comprises a T cell line. In some embodiments, the plurality of cells comprises Jurkat T cells.

In some embodiments, one or more of the plurality of cells is cultured on one or more of the compositions as described herein. In some embodiments, the plurality of cells remains viable for at least 4 days of culturing. In some embodiments, the plurality of cells remains viable for at least 6 days of culturing. In some embodiments, the plurality of cells remains viable for at least 10 days of culturing.

### Methods of Production

Methods of producing compositions of the present invention comprise: providing a piezoelectric polymer scaffold, providing a plurality of charged nanoparticles, providing a buffer solution, combining the piezoelectric polymer scaffold, plurality of charged nanoparticles, and the buffer solution, and applying a vacuum to the buffer solution, and repeatedly agitating the buffer solution, so as to thereby so as to thereby non-covalently adsorb the plurality of charged nanoparticles onto the piezoelectric polymer scaffold. In some embodiments, the plurality of charged nanoparticles are electrostatically adsorbed onto the piezoelectric polymer scaffold.

In some embodiments, the methods further comprise electrospinning a piezoelectric polymer scaffold.

In some embodiments, the buffer solution comprises HEPES buffer solution. In some embodiments, the amount of buffer is from 100 mM to 1000 mM. In some embodiments, the amount of buffer is from 250 mM to 750 mM. In some embodiments, the amount of buffer is 500 mM

In some embodiments, the buffer solution has a Debye electrostatic screening length of less than 0.700 nm. In some embodiments, the buffer solution has a Debye electrostatic screening length of less than 0.500 nm. In some embodiments, the buffer solution has a Debye electrostatic screening length of less than 0.250 nm.

In some embodiments, the piezoelectric polymer scaffold is electrospun.

In some embodiments, the pH of the buffer solution is 5-9. In some embodiments, the pH of the buffer solution is 6-8. In some embodiments, the pH of the buffer solution is 6.5-8. In some embodiments, the pH of the buffer solution is 6.8-7.7. In some embodiments, the pH of the buffer solution is 6.9-7.6. In some embodiments, the pH of the buffer solution is 7.5.

In some embodiments, the ionic strength of the buffer solution is at least 0.4 M. In some embodiments, the ionic strength of the buffer solution is at least 0.8 M. In some embodiments, the ionic strength of the buffer solution is at least 1 M.

In some embodiments, the buffer solution comprises a salt. In some embodiments, the salt comprises NaCl. In some embodiments, the amount of salt is from 100 mM to 1000 mM. In some embodiments, the amount of salt is from 250 mM to 750 mM. In some embodiments, the amount of salt is from 400 mM to 600 mM. In some embodiments, the amount of salt is 500 mM.

In some embodiments, the concentration of the charged nanoparticles in the buffer solution is at least 0.1 mg/mL. In some embodiments, the concentration of the charged nanoparticles in the buffer solution is at least 1 mg/mL. In some embodiments, the concentration of the charged nanoparticles in the buffer solution is at least 2 mg/mL.

### Methods of Use

In some embodiments, methods of use the present invention include use of compositions of the present invention. All compositions of the present invention can be used in the methods of use.

In some embodiments, compositions according to the present invention are used for improving tissue engineering. In some embodiments, compositions comprise a plurality of mammalian epithelial, endothelial, stromal, mesenchymal, parenchymal, progenitor, stem, and/or differentiated cells and the composition is used in a tissue engineering environment, thereby improving the tissue engineering. In some embodiments, compositions comprise a plurality of human epithelial, endothelial, stromal, mesenchymal, parenchymal, progenitor, stem, and/or differentiated cells and the composition is used in a tissue engineering environment, thereby improving the tissue engineering.

In some embodiments, compositions according to the present invention are used for improving immune engineering. In some embodiments, compositions comprise a plurality of mammalian immune, activated, or engineered cells and the composition is used in an immune engineering environment, thereby improving the immune engineering. In some embodiments, compositions comprise a plurality of human immune, activated, or engineered cells and the composition is used in an immune engineering environment, thereby improving the immune engineering.

In some embodiments, the plurality of cells actively engage and/or take up components of the plurality of charged nanoparticles. In some embodiments, the plurality of cells actively engage and/or take up components of liposomes.

In some embodiments, the present invention comprise a composition comprising: a piezoelectric polymer scaffold, wherein the piezoelectric polymer scaffold is electrospun; and a plurality of charged nanoparticles, wherein the plurality of charged nanoparticles are non-covalently adsorbed onto the piezoelectric polymer scaffold for use in would healing, repairing nerve gaps, or cell culture, wherein the wound healing, repairing nerve gaps, or cell culture is improved by the use of the composition.

In some embodiments, the present invention provides a method of local administration of a drug comprising: providing a composition comprising: a piezoelectric polymer scaffold, wherein the piezoelectric polymer scaffold is electrospun; and a plurality of charged nanoparticles, wherein the plurality of charged nanoparticles are non-covalently adsorbed onto the piezoelectric polymer scaffold, and the drug is bonded to the charged nanoparticles; and applying the composition in a drug administration environment, thereby improving the local administration of the drug.

In some embodiments, the present invention provides a method of treating a wound comprising: providing a composition comprising: a piezoelectric polymer scaffold, wherein the piezoelectric polymer scaffold is electrospun; and a plurality of charged nanoparticles, wherein the plurality of charged nanoparticles are non-covalently adsorbed onto the piezoelectric polymer scaffold; and applying the composition to the site of a wound, thereby improving the treatment of the wound.

In some embodiments, the present invention provides a method of modulating activity in a tissue comprising: providing a composition comprising: a piezoelectric polymer scaffold, wherein the piezoelectric polymer scaffold is electrospun; and a plurality of charged nanoparticles, wherein the plurality of charged nanoparticles are non-covalently adsorbed onto the piezoelectric polymer scaffold; and applying the composition to a tissue, thereby modulating the tissue. In some embodiments, the plurality of charged nanoparticles are electrostatically adsorbed onto the piezoelectric polymer scaffold. In some embodiments, the tissue is CNS tissue, spinal cord tissue, peripheral nervous system tissue, bone tissue, cartilage tissue, bone marrow, or other human organ tissue.

In some embodiments, the present invention provides a method of treating or reducing inflammation comprising: providing a composition comprising: a piezoelectric polymer scaffold, wherein the piezoelectric polymer scaffold is electrospun; and a plurality of charged nanoparticles, wherein the plurality of charged nanoparticles are non-covalently adsorbed onto the piezoelectric polymer scaffold; and applying the composition to a site of inflammation, thereby treating or reducing inflammation. In some embodiments, the inflammation is decreased or treated in a plurality of cells. In some embodiments, the plurality of cells are immune cells. In some embodiments, the plurality of charged nanoparticles are functionalized with anti-inflammatory cytokines.

In some embodiments, the present invention provides a method improving cell culture comprising: providing a composition comprising: a piezoelectric polymer scaffold, wherein the piezoelectric polymer scaffold is electrospun; and a plurality of charged nanoparticles, wherein the plurality of charged nanoparticles are non-covalently adsorbed onto the piezoelectric polymer scaffold; and applying the composition to cell culture environment, thereby improving the cell culture. In some embodiments, the cell culture is ex-vivo. In some embodiments, the cell culture is in-vitro.

In some embodiments, the piezoelectric polymer scaffold comprises a piezoelectric fluoropolymer scaffold. In some embodiments, the piezoelectric polymer scaffold is electrospun. In some embodiments, the plurality of charged nanoparticles are electrostatically adsorbed onto the piezoelectric polymer scaffold. In some embodiments, the plurality of charged nanoparticles densely and/or uniformly coat the electrospun piezoelectric polymer scaffold. In some embodiments, the plurality of charged nanoparticles comprises functionalized polymer latex nanoparticles, layer-by-layer coated nanoparticles, and/or liposomes. In some embodiments, the plurality of charged nanoparticles comprise functionalized charged nanoparticles comprising an active agent, wherein the active agent comprises one or more of: a protein, an antibody or fragment thereof, a single-chain variable fragment, a chimeric antigen receptor, or a nucleic acid. In some embodiments, one or more of the active agents is encapsulated by a functionalized charged nanoparticle and/or is surface mounted to one or more of the functionalized charged nanoparticles. In some embodiments, the active agent is surface mounted via a metal-chelating lipid. In some embodiments, the plurality of charged nanoparticles comprises liposomes and an immune modulator, wherein the immune modulator is surface mounted to one or more of the plurality of charged nanoparticles via a metal-chelating lipid. In some embodiments, the composition further comprises a plurality of biological cells adhered to the piezoelectric polymer scaffold and/or one or more of the plurality of charged nanoparticles or associated therewith. In some embodiments, the composition further comprises a plurality of biological cells, wherein the plurality of biological cells comprises mammalian cells. In some embodiments, the plurality of biological cells actively engages with and/or takes up components of the plurality of charged nanoparticles.

### ADDITIONAL EMBODIMENTS

The disclosed subject matter provides methods for enhancing the topographical and/or piezoelectric properties of electrospun scaffolds by integrating cytokines-tethered nanoparticles. These surface-functionalized electrospun scaffolds can introduce potent anti-inflammatory cues into the system and direct the phenotype of astrocytes within CNS lesions to facilitate effective spinal cord regeneration.

In some embodiments, the disclosed subject matter provides a method of charging the nanoparticles so that they are electrostatically adsorbed onto electrospun scaffolds. In some embodiments, the nanoparticles are charged by surface modification. In some embodiments, the nanoparticles are charged by coating charged layers. In some embodiments, the nanoparticles are charged by incorporating charged moieties or molecules.

In some embodiments, the disclosed subject matter provides a method of charging electrospun scaffolds through corona poling. In some embodiments, the electrospun scaffolds are made from polyvinylidene-trifluoroethylene (PVDF-TrFE) copolymer. In some embodiments, the PVDF-TrFE scaffolds are positively charged so that negatively charged nanoparticles are electrostatically adsorbed to the surface of the scaffolds. In some embodiments, the PVDF-TrFE scaffolds are negatively charged so that positively charged nanoparticles are electrostatically adsorbed to the surface of the scaffolds.

In some embodiments, the disclosed nanoparticles are made from latex and surface modified with negatively charged groups, such as carboxy groups. In some embodiments, the size of these carboxyl-modified latex (CML) nanoparticles ranges from 70 nm to 200 nm, preferably 100 nm to 150 nm and preferably around 130 nm. The charge of these nanoparticles is negative. In some embodiments, the latex nanoparticles are made from polystyrene.

In some embodiments, the disclosed nanoparticles are layer-by-layer (LbL) coated with polymers. Each individual layer is either positively charged or negatively charged and assembled to form a multiple-layer structure. In some embodiments, the positively charged layer comprises Poly(allylamine hydrochloride) (PAH+). In some embodiments, the negatively charged layer comprises Poly(acrylic acid) (PAA−). In some embodiments, the plurality of charged nanoparticles can further incorporate adjuvants, nucleic acids, and other functional biopolymers. In certain example embodiments, the size of these LbL nanoparticles is above 100 nm. In some embodiments, the size of these LbL nanoparticles is from 10-900 nm. In some embodiments, the size of these LbL nanoparticles is from 50-200 nm. In some embodiments, the size of these LbL nanoparticles is from 100-150 nm. In some embodiments, the charge of these LbL nanoparticles is positive. In some embodiments, the charge of these LbL nanoparticles is negative.

In some embodiments, the disclosed nanoparticles are made from liposomes with charged moieties or molecules. In some embodiments, the charged liposome nanoparticles comprise nitrilotriacetic acid (NTA), a chelating agent that binds to metal ions-such as nickel (Ni) ions. In some embodiments, the charged liposome nanoparticles form a stable NTA-Ni complex and have a specific affinity for histidine tags (e.g., polyhistidine or His-tags). In some embodiments, the charged liposome-NTA-Ni complex tethers a growth factor (e.g., cytokines) via 6× His-tag sandwiched between the complex and the growth factor. In some embodiments, the liposome in the complex is positively charged.

In some embodiments, the size of the disclosed liposome nanoparticles ranges from below 100 nm to 1000 nm, preferably around 150 nm. In some embodiments, the size of the disclosed liposome nanoparticles ranges from 100-500 nm. In some embodiments, the size of the disclosed liposome nanoparticles ranges from 100-200 nm. In some embodiments, the charge of these nanoparticles is positive. In some embodiments, the charge of these nanoparticles is negative. In some embodiments, the disclosed liposome comprises phospholipids, such as DTAP, DSPC, and cholesterol. In some embodiments, the existence of liposome coatings on the scaffolds can be evaluated by an energy-dispersive X-ray spectroscopy. An increase in the content of nitrogen can be observed due to the presence of amino groups on the phospholipid components of the adsorbed cationic liposomes on the scaffolds.

In some embodiments, the disclosed subject matter provides a method of tuning the extent of the disclosed nanoparticle coatings on the electrospun scaffolds. In some embodiments, the tuning comprises adjusting the pH and ionic strength of loading solutions of the nanoparticles. In certain example embodiments, HEPES and NaCl are added to the loading solutions.

In some embodiments, electron microscopy can be used to reveal the density of the coatings in different loading conditions. In some embodiments, confocal microscopy can be used to reveal the coating of each type of disclosed nanoparticles by fluorescently labeling the nanoparticles. In some embodiments, IL-10 tethered liposomes are tested to evaluate their coating density on the disclosed scaffolds.

In some embodiments, the coating density of LbL nanoparticles increases as the loading concentration increases. In some embodiments, 2 mg/mL of LbL nanoparticles can achieve a near-complete coating of the scaffolds in distilled H₂O (dH₂O) as compared to HEPES and HEPES-NaCl solutions. In certain example embodiments, 0.5 mg/mL of LbL nanoparticles can achieve a near complete coating of the scaffolds in HEPES, as compared to HEPES-NaCl and dH₂O.

In some embodiments, the coating density of unlayered CML nanoparticles in different loading solutions is evaluated. In certain example embodiments, 2 mg/mL of the unlayered CML nanoparticles can achieve a near-complete coating of the scaffolds in HEPES-NaCl as compared to HEPES and dH₂O. In certain example, embodiments, 2 mg/mL of the liposome nanoparticles can achieve a half-complete coating of the scaffolds in all three loading solutions (i.e., HEPES-NaCl, HEPES, and dH₂O).

In some embodiments, the techniques described herein relate to a composition including: (a) a piezoelectric polymer scaffold; and (b) a plurality of charged nanoparticles, non-covalently adsorbed onto the piezoelectric polymer scaffold.

In some embodiments, the techniques described herein relate to a composition, wherein the piezoelectric polymer scaffold includes a piezoelectric fluoropolymer scaffold.

In some embodiments, the techniques described herein relate to a composition, wherein the piezoelectric polymer scaffold is electrospun.

In some embodiments, the techniques described herein relate to a composition, wherein the plurality of charged nanoparticles are electrostatically adsorbed onto the piezoelectric polymer scaffold.

In some embodiments, the techniques described herein relate to a composition, wherein when the plurality of charged nanoparticles is fluorescently labeled, and the fluorescence is measured via an inverted confocal fluorescence microscope, the mean fluorescence intensity density per micron of the piezoelectric polymer scaffold is from 20 RFU/μm²-40 RFU/μm².

In some embodiments, the techniques described herein relate to a composition, wherein the plurality of charged nanoparticles includes functionalized polymer latex nanoparticles, layer-by-layer coated nanoparticles, and/or liposomes.

In some embodiments, the techniques described herein relate to a composition, wherein the plurality of charged nanoparticles include functionalized charged nanoparticles including an active agent, wherein the active agent includes one or more of: a protein, an antibody or fragment thereof, a single-chain variable fragment, a chimeric antigen receptor, or a nucleic acid.

In some embodiments, the techniques described herein relate to a composition, wherein one or more of the active agents is encapsulated by a functionalized charged nanoparticle and/or is surface mounted to one or more of the functionalized charged nanoparticles.

In some embodiments, the techniques described herein relate to a composition, wherein the active agent is surface mounted via a metal-chelating lipid.

In some embodiments, the techniques described herein relate to a composition, wherein the plurality of charged nanoparticles includes liposomes and an immune modulator, optionally wherein the immune modulator is surface mounted to one or more of the plurality of charged nanoparticles via a metal-chelating lipid.

In some embodiments, the techniques described herein relate to a composition, further including a plurality of biological cells adhered to the piezoelectric polymer scaffold and/or one or more of the plurality of charged nanoparticles or associated therewith.

In some embodiments, the techniques described herein relate to a composition, wherein the composition further includes a plurality of biological cells, optionally wherein the plurality of biological cells includes mammalian cells.

In some embodiments, the techniques described herein relate to a method 1, so as to thereby modulate activity of the tissue.

In some embodiments, the techniques described herein relate to a method of improving three dimensionality of a cell culture including: (a) providing a composition including: (i) a piezoelectric polymer scaffold; and (ii) a plurality of charged nanoparticles, non-covalently adsorbed onto the piezoelectric polymer scaffold; (b) applying the composition into a cell culture environment, thereby improving the three dimensionality of the cell culture.

In some embodiments, the techniques described herein relate to a method, wherein the cell culture is an immune cell culture and the nanoparticles include one or more immune modulators.

In some embodiments, the techniques described herein relate to a method of producing a composition including: (a) providing a piezoelectric polymer scaffold, wherein the piezoelectric polymer scaffold is electrospun; (b) providing a plurality of charged nanoparticles; (c) providing a buffer solution; (d) combining the piezoelectric polymer scaffold, the plurality of charged nanoparticles, and the buffer solution; (e) applying a vacuum to the buffer solution, and repeatedly agitating the buffer solution, so as to thereby non-covalently adsorb the plurality of charged nanoparticles onto the piezoelectric polymer scaffold and produce the composition.

In some embodiments, the techniques described herein relate to a method, wherein the piezoelectric polymer scaffold is corona poled.

In some embodiments, the techniques described herein relate to a method, wherein the pH of the buffer solution is 5-9.

In some embodiments, the techniques described herein relate to a method, wherein the ionic strength of the buffer solution is at least 0.4 M.

In some embodiments, the techniques described herein relate to a method, wherein the concentration of the plurality of charged nanoparticles in the buffer solution is at least 0.1 mg/mL.

### EXAMPLES

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1—Tunable Electrostatic Adsorption of Polymeric Nanoparticles onto Piezoelectric Scaffolds

Electrostatic self-assembly offers a versatile strategy for surface functionalization, as exemplified by layer-by-layer (LbL) techniques that use alternating charged species to build well-defined interfaces.⁴¹ While these approaches have been widely applied to planar or chemically modified substrates,⁴²⁻⁴⁴ their applications to three-dimensional, electrospun fibrous scaffolds is less explored. It was hypothesized that the intrinsic surface charge of PVDF-TrFE,⁴⁵ a piezoelectric polymer, which when annealed has a voltage output of approximately 1V/mm⁴⁶ might be able to support electrostatic nanoparticle adsorption without requiring covalent modification. To test this, it was investigated how nanoparticle composition and coating method influence electrostatic adsorption onto these piezoelectric substrates.

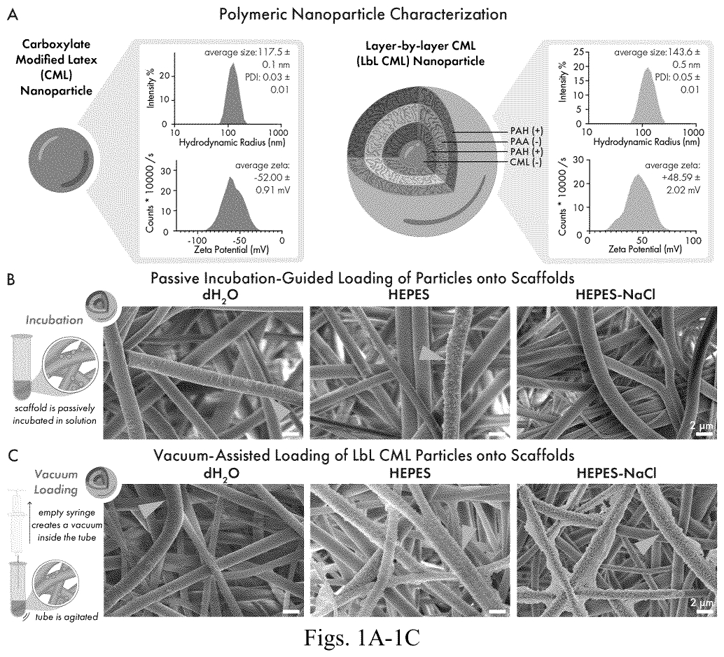

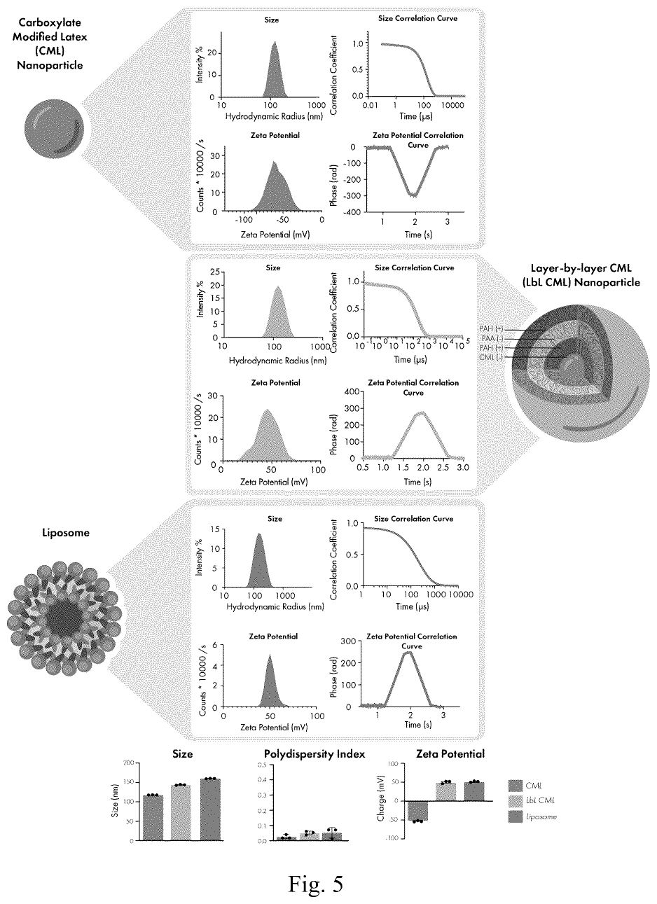

To explore how nanoparticle surface properties influence scaffold adsorption, two nanoparticle types were compared: commercial carboxylate-modified latex (CML) particles, which are stiff and smooth, and LbL-coated CML nanoparticles, which are expected to be softer and have a rougher surface due to their loopy, entangled polymer layers.⁴⁶ Cationic LbL nanoparticles were generated by sequentially coating anionic CML particles with three polyelectrolyte layers: cationic poly-allylamine hydrochloride (PAH), anionic poly(acrylic acid) (PAA), and a final layer of PAH. This multilayered coating increased the particle diameter from 117.4±0.1 nm to 143.6±0.5 nm, as measured by dynamic light scattering. Both particle types displayed narrow size distributions (PDI=0.03±0.01 or 0.05±0.01, respectively), and their high-magnitude zeta potentials (−52.00±0.91 mV or ±48.59±2.02 mV, respectively) (FIG. 5A) confirmed strong surface charge, making them suitable for electrostatic adsorption (FIG. 1A).

To generate a suitable substrate for electrostatic adsorption, PVDF-TrFE scaffolds were synthesized and confirmed to have a fiber diameter of 1.80±0.51 μm using Fiji-ImageJ analysis of images taken using scanning electron microscopy (SEM), consistent with previous reports of electrospun PVDF-TrFE scaffold fiber diameters.^{47,48} To facilitate electrostatic adsorption of nanoparticles, the substrate must carry a strong surface charge of opposite polarity to the adsorbing species.³⁵ Piezoelectric materials like PVDF-TrFE offer a unique advantage in this context: they can generate surface charge intrinsically through the alignment of molecular dipoles.^{45,49,50} This property can be harnessed in two ways. First, stable charge domains can be established through corona poling, in which the scaffold is exposed to a high-voltage electric field to induce long-range dipole alignment and persistent polarization.^{47,51,52} Second, mechanical deformation can induce local dipole reorientation and transient electric fields, activating surface charge in a dynamic and spatially heterogeneous manner.^{53,54,49}

To optimize scaffold coating, how these two charge-generating mechanisms influence nanoparticle adsorption was evaluated by comparing two nanoparticle loading strategies using corona-poled scaffolds: (i) vacuum-assisted deposition and (ii) passive incubation. The vacuum-assisted method was designed to introduce mechanical stress during coating by placing scaffolds in a sealed tube filled with nanoparticle solution. Within this tube, negative pressure was applied via syringe, and the tube was repeatedly agitated to induce scaffold flexion. In contrast, in the passive incubation method, scaffolds were submerged in nanoparticle solution without agitation. SEM revealed that passive incubation-based loading resulted in minimal nanoparticle adsorption onto the scaffold, whereas vacuum-assisted loading produced significantly denser and more uniform coatings (FIG. 1B).

Although it was observed that mechanical activation enhanced nanoparticle adsorption, the overall coating density remained low across tested conditions. It was hypothesized that, beyond surface charge effects, adsorption efficiency may also depend on solution conditions known to modulate electrostatic interactions—a well-established principle in layer-by-layer electrostatic assembly.^{34,55,56} To test this, it was investigated how solution pH and ionic strength influence nanoparticle binding, with the goal of optimizing coating conditions to achieve a thicker and denser coating.

Scaffold coating was evaluated in three buffer systems designed to independently modulate pH and ionic strength. Buffer pH was selected to stabilize the ionization states of charged functional groups on the nanoparticle surface. CML nanoparticles bear-COOH groups (pKa ~4.5-5.0) that are fully deprotonated and negatively charged above pH 6, while PAH, used as the terminal layer on LbL nanoparticles, contains primary amines (pKa ~8.5-9.3) that remain protonated and positively charged below this range. HEPES buffer (500 mM, pH 7.5, ionic strength=0.5M, Debye electrostatic screening length λ_{D} D=0.430 nm) was chosen to ensure strong ionization of both groups and minimize pH drift during loading. Zwitterionic buffers such as HEPES exhibit the unique property of not contributing meaningfully to the solution's ionic strength, which allows us to tune pH without impacting the Debye electrostatic screening length. In parallel, ionic strength was tuned to regulate interparticle and particle-scaffold electrostatic interactions: low ionic strength minimizes charge screening but can increase repulsion between similarly charged species, while moderate ionic strength can reduce repulsion and promote closer packing.

To create these conditions, deionized water was used as a low-ionic strength, unbuffered condition. HEPES supplementation offered pH control with negligible ionic strength.³⁵ Lastly, 500 mM HEPES supplemented with 400 mM NaCl introduced a moderate ionic strength (ionic strength=0.9M, Debye electrostatic screening length λ_{D}=0.320 nm) to enhance adsorption. Each condition was tested at a high and low nanoparticle concentration (2 mg/mL and 0.5 mg/mL), alongside buffer-only vacuum-loaded controls. SEM revealed that when employing vacuum-assisted loading, scaffolds treated in HEPES-NaCl exhibited the most uniform coverage, had moderate plaque-like adsorption of particles in HEPES alone, and were sparsely and inconsistently coated by particles in deionized water (FIG. 1C). These findings indicate that optimal adsorption required simultaneous tuning of both pH and ionic strength to support charge stability and minimize repulsive interactions. However, in the passive incubation condition, modest nanoparticle coverage was observed when loading in HEPES buffer alone, but little to no coverage occurred in HEPES supplemented with NaCl (FIG. 1B). It was hypothesized that in the absence of mechanical agitation, the scaffold fibers possess low surface charge, and that the addition of salt leads to near-complete electrostatic charge screening that prevents interactions between nanoparticles and the scaffold.

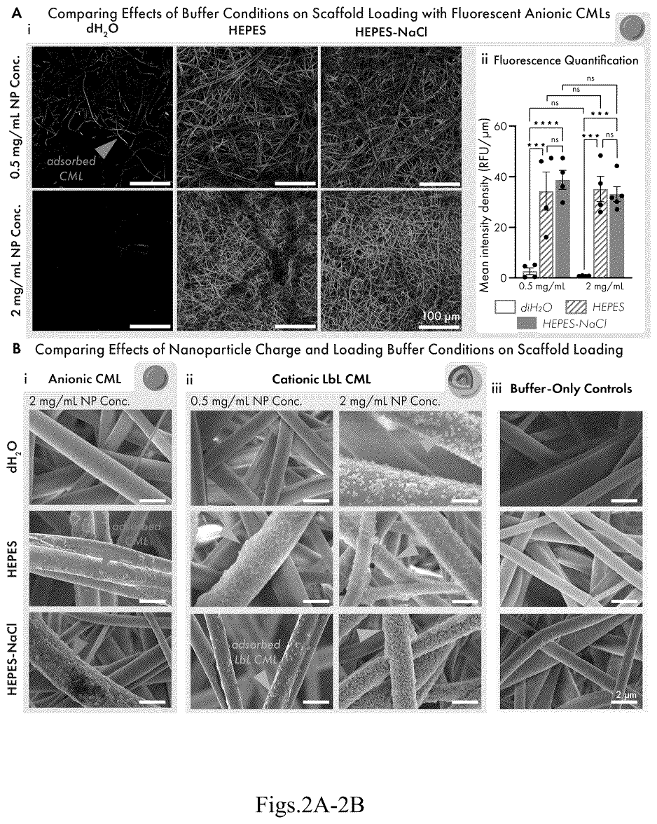

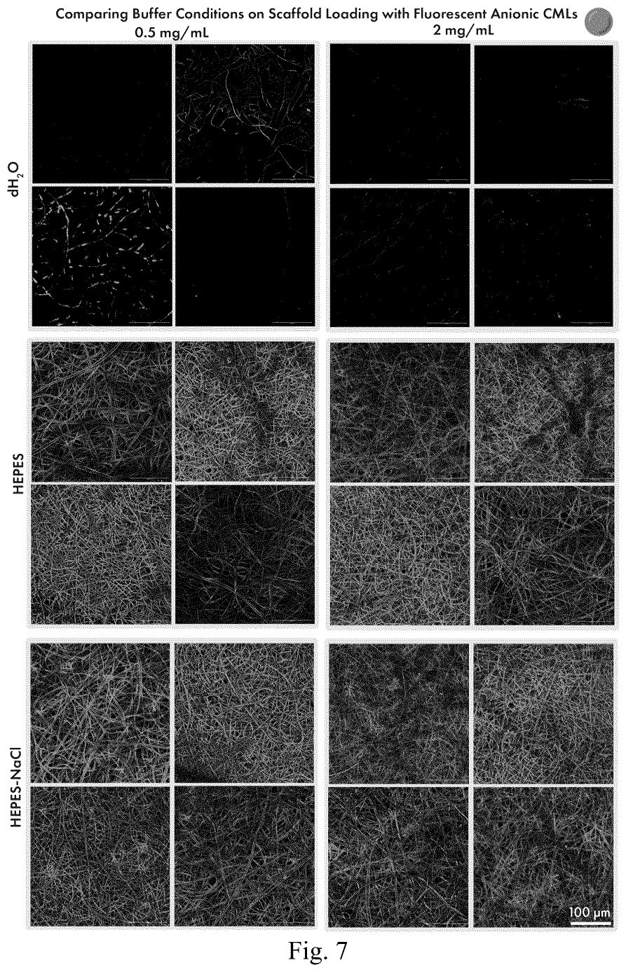

Next the effects of buffer composition of scaffold loading using fluorescent anionic CMLs were validated, enabling the quantitative comparison of both loading efficiency and reproducibility across conditions and nanoparticle densities in a larger field of view. Consistent with the high-resolution SEM that informed morphological understanding of NP adsorption, confocal imaging revealed that HEPES-NaCl produced both greater loading efficiency and more uniform coating across larger scaffold regions (FIG. 2A panel i, FIG. 7). The mean fluorescence intensity density per micron at high (2 mg/mL) NP concentration was 33.12±2.94 RFU/μm² in HEPES-NaCl, 35.19±5.01 RFU/μm² in HEPES, and 0.74±0.07 RFU/μm² in deionized water, and at low (0.5 mg/mL) NP concentration was 38.76±3.76 RFU/μm² in HEPES-NaCl, 34.33±7.54 RFU/μm² in HEPES, and 2.60±1.31 RFU/μm² in deionized water (FIG. 2 A panel ii).

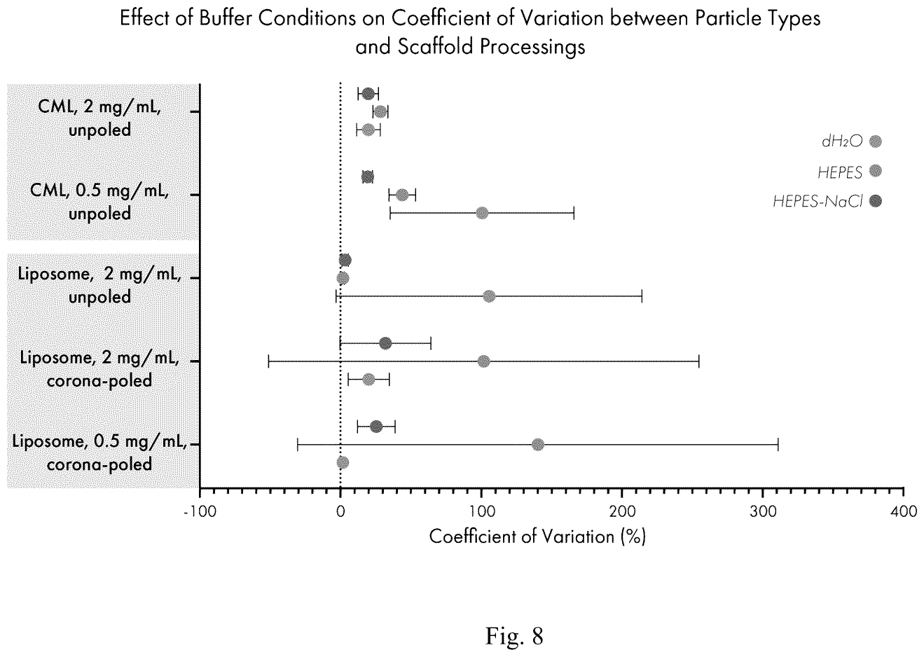

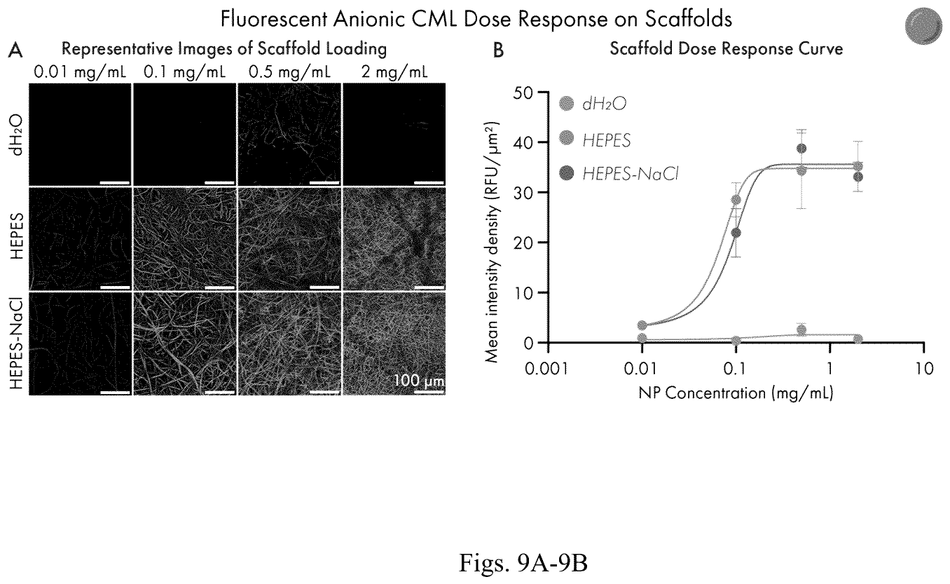

Inclusion of NaCl appeared to improve coating consistency, as indicated by a smaller coefficient of variation at high NP concentration (19.83±7.23% in HEPES-NaCl compared to 28.48±5.32% in HEPES and 19.92±8.37% in deionized water) and at low NP concentration (19.41±3.41% in HEPES-NaCl compared to 43.92=9.44% in HEPES and 100.63±65.20% in deionized water) (FIG. 8). While significant benefit was seen in coating density and reproducibility by modulating buffer pH and ionic strength, significant differences were not observed in coating density between high and low nanoparticle concentrations in the same buffer (Table 1), suggesting that scaffolds were becoming saturated at the low NP concentration. By repeating this experiment across lower NP concentrations, it was identified that scaffold saturation occurs between 0.1 and 0.5 mg/mL CML concentration in HEPES and HEPES-NaCl (FIG. 9). This data indicates that while low NP concentrations may approach scaffold saturation under favorable conditions, increased particle concentration promotes more uniform and reproducible adsorption.

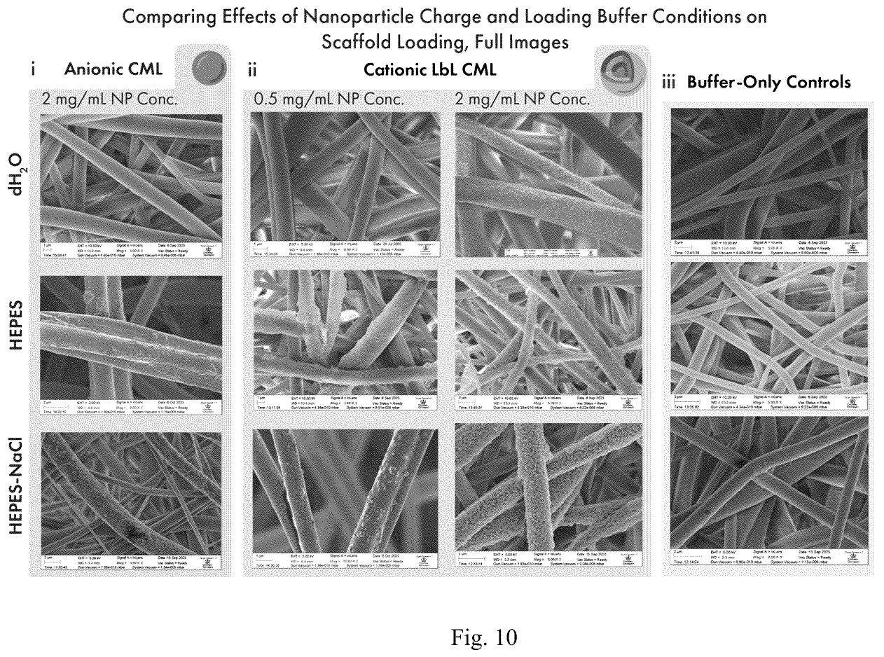

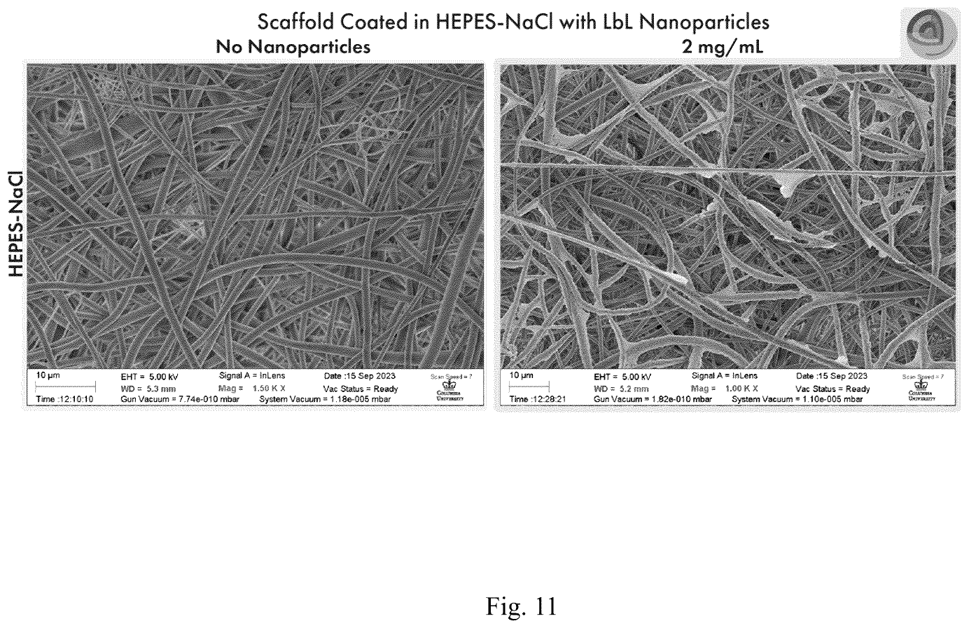

The effect of particle charge on adsorption was investigated. Notably, particle adsorption was observed for both cationic and anionic nanoparticles (FIG. 2 B panels i, ii), heterogeneously coating fibers throughout the scaffold (FIGS. 10, 11). This suggests that the bulk scaffold system does not carry a single dominant charge, rather that the fibrous piezoelectric architecture generates heterogeneous surface potentials during mechanical deformation, with coexisting microdomains of positive and negative charge. This is consistent with prior studies showing that strain gradients in piezoelectric polymers, particularly in thin and flexible materials, can produce spatially patterned electric fields.⁵⁷ Several groups have leveraged this behavior to guide polymer adsorption or create self-patterned materials using electrostatic self-assembly principles.⁵⁸⁻⁶⁰

Together, these results introduce a strategy for non-covalent, post-fabrication functionalization of piezoelectric scaffolds with nanotechnology. This is significant for tissue engineering applications, where electrospun fibers are commonly used as extracellular matrix (ECM) mimics^{26,61}. While strategies such as physical adsorption or encapsulation of proteins and small molecules,⁶²⁻⁶⁵ blend electrospinning of polymers with bioactive agents,^{30,66-68} coaxial electrospinning for core-shell architectures,⁶⁹⁻⁷¹ and covalent immobilization of ligands or growth factors,⁷²⁻⁷⁴ have all been used to functionalize scaffolds, they often involve harsh processing, irreversible chemistry, or limited modularity.⁷⁵ In contrast, the electrostatic adsorption method in this application circumvents many of these issues by enabling post-fabrication scaffold modification under mild conditions, with tunable coating density using either cationic or anionic nanoparticles. This render the techniques particularly well-suited for presenting fragile or multi-component nanocarriers and supports its utility in modular regenerative medicine platforms.

FIGS. 1A-1C depict systematic comparison of loading methodology on charged particles which reveals optimal loading technique. FIG. 1A shows carboxylate modified latex (CML) particle (left) and layer-by-layer CML particles (LbL CML, right). Hydrodynamic radius and zeta potential are characterized with dynamic light scattering; values are reported as mean±SEM with n=3 replicates. FIG. 1B shows scanning electron microscopy images of particle coatings with incubation-guided loading in deionized water (left), HEPES buffer (middle), or HEPES buffer+NaCl with LbL CML NPs at 2 mg/mL. FIG. 1C shows scanning electron microscopy images of particle coatings with vacuum-assisted loading in deionized water (left), HEPES buffer (middle), or HEPES buffer+NaCl with LbL CML NPs at 2 mg/mL. Arrows indicate discrete particles and/or particle plaques.

FIGS. 2A-2C depict how solution conditions tune nanoparticle loading onto scaffolds. FIG. 2A panel I shows confocal microscopy of 50 μm Z-stack maximum intensity projections of adsorbed CMLs on unpoled scaffolds at different densities (0.5 mg/mL and 2 mg/mL) in dH2O, HEPES, and HEPES-NaCl (left to right), scale bar=100 μm and is applicable to all images; panel ii shows image-based quantification of mean fluorescence intensity of unpoled coated scaffolds with n=4 scaffolds/condition. Two-way ANOVA is followed by Šídák's multiple comparisons test (*p<0.05, ** p<0.01, *** p<0.001, **** p<0.0001). FIG. 2B shows scanning electron microscopy images of CML or LbL CML particle coatings at different densities (0.5 mg/mL and 2 mg/mL) and in different loading buffers (dH2O, HEPES, HEPES-NaCl (top to bottom)) on corona poled electrospun scaffolds: panel i CML, panel ii LbL CML, and panel iii no particles. Arrows indicate discrete particles and/or particle plaques. Scale bar=2 μm and is applicable to all images. Values are represented as mean±SEM.

### Example 2—Extending Nanomaterial Coatings to Liposomal Nanotechnology

To adapt the nanoparticle coating platform for the delivery of biologically active signals, liposomes were selected as a modular and clinically relevant nanocarrier. Liposomes are biocompatible vesicles that can encapsulate small molecules within their bilayer or aqueous core, and their surfaces can be engineered to display proteins, lipids, or targeting ligands.⁷⁶⁻⁷⁹ Their surface chemistry, including overall charge, can be readily tuned by selecting different phospholipids and lipid components.⁸⁰ By varying these parameters, liposomes can be engineered to support both electrostatic adsorption to the scaffold and programmable display of bioactive signals at the interface, making them a compelling platform for functionalizing scaffolds.

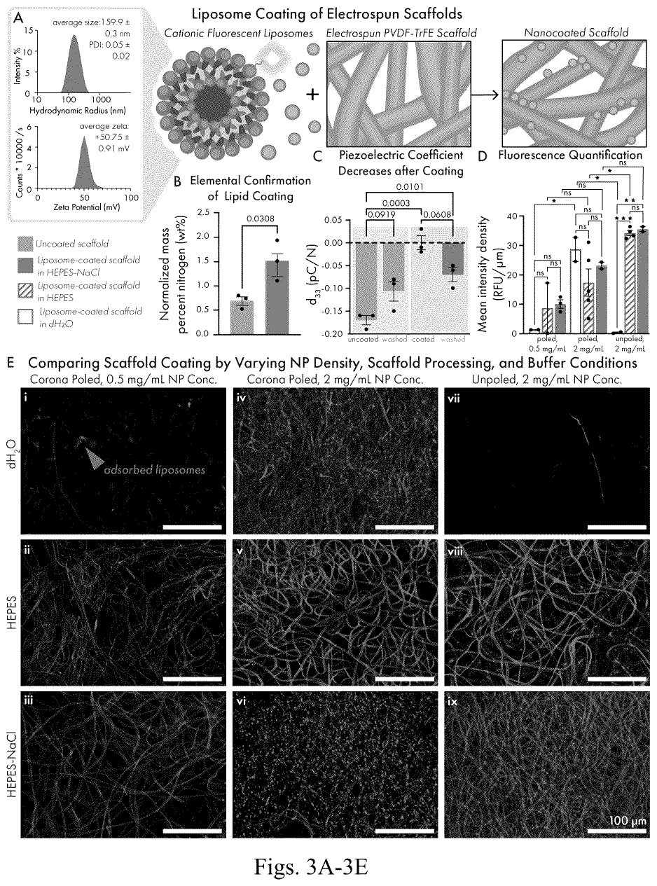

Building on the results with polymeric nanoparticles, it was hypothesized that charged liposomes could similarly adsorb to the scaffold under optimized buffer conditions. A cationic liposome formulation consisting of 1,2-distearoyl-sn-glycero-3-phosphocholine (DSPC), 1,2-dioleoyl-3-trimethylammonium-propane (DOTAP), and plant cholesterol, a 9:1:2: molar ratio, was selected which was based on previous work with liposomal biomaterials for drug delivery,⁷⁹ and to promote adhesion to the negatively charged glycocalyx on mammalian cells.^{81,82} This design was intended to preserve scaffold-bound liposome accessibility and enhance cell-material interactions. Unilamellar liposomes were synthesized with a mean diameter of 159.9±0.3 nm, a low polydispersity index (0.05±0.02), and a zeta potential of ±50.75±0.91 mV, indicating a stable, highly charged nanoparticle suitable for electrostatic self-assembly.^{76,83} (FIG. 3A).

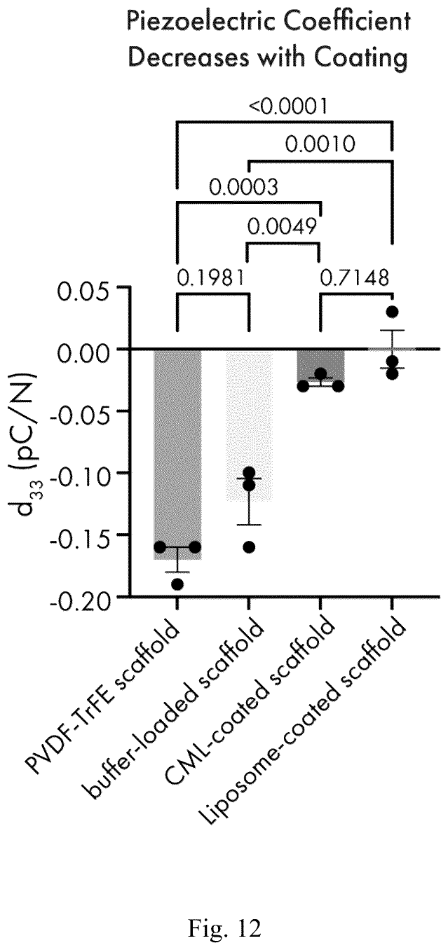

To initially validate liposome adsorption, scaffolds were coated using the optimal HEPES-NaCl buffer conditions we identified in the polymeric nanoparticle experiments. SEM combined with energy-dispersive X-ray spectroscopy (EDS) was used to detect levels of elemental nitrogen, which is present in the DSPC and DOTAP components of the liposome formulation, but is absent from the PVDF-TrFE scaffold itself. EDS confirmed that nitrogen levels were significantly higher in liposome-coated scaffolds compared to uncoated scaffolds (1.522±0.291% vs. 0.692±0.107%, p=0.03080), indicating successful adsorption (FIG. 3 B). It was then explored whether nanoparticle coatings influenced the intrinsic piezoelectric properties of the scaffold. To assess this, the piezoelectric coefficient (d₃₃) of PVDF-TrFE scaffolds was measured before and after liposome coating. Scaffolds showed a significant (p=0.0003) reduction in piezoelectric response after coating, with the magnitude of the d₃₃ value decreasing from −0.17±0.01 pC/N in uncoated scaffolds to 0.00±0.02 pC/N following liposome adsorption (FIG. 3 C). This effect was also observed with polymeric nanoparticles (FIG. 12). These findings suggest that charged nanoparticle adsorption may attenuate piezoelectric behaviors in the hybrid system. However, this piezoelectric shielding effect is recovered when lipids are removed from the scaffold with ethanol, restoring the d₃₃ value to −0.07±0.02 pC/N on washed scaffolds. While this restored d₃₃ is not significantly different from that of liposome-coated scaffolds (p=0.0608), the large effect size (Cohen's d=2.65) in conjugation with its strong trend suggest an apparent reduction in piezoelectric coefficient, rather than a true change in the intrinsic properties of the PVDF-TrFE fibers, due to the presence of a coating layer.

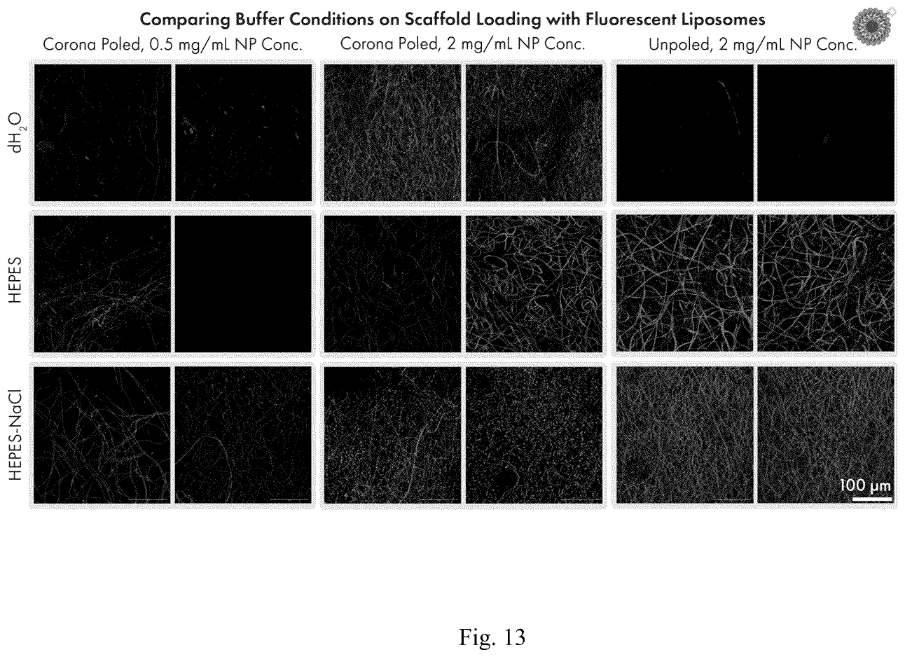

To determine the tunability of liposome coatings, the extent of adsorption could be modulated by solution conditions was explored. This allowed testing whether the design rules observed for polymeric nanoparticle coatings, specifically the roles of pH and ionic strength, extended to lipid-based nanocarriers. The vacuum-assisted loaded technique was used to adsorb fluorescently labeled liposomes (Cy5 liposomes) onto scaffolds using our three loading solutions: deionized water, HEPES buffer (500 mM, pH=7.5), and HEPES (500 mM) further supplemented with NaCl (400 mM). Confocal microscopy revealed differences in coating density and uniformity on corona-poled scaffolds that were consistent with the results with polymeric nanoparticles: the most consistently dense coatings across NP concentrations were observed when using HEPES-NaCl (19.40±1.24 RFU/μm² at high NP concentration, 10.01±0.85 RFU/μm² at low NP concentration), followed by HEPES alone (18.07±9.20 RFU/μm² at high NP concentration, 8.64 £ 6.06 RFU/μm² at low NP concentration), and scaffolds coated in deionized water displayed sparse and heterogeneous coatings (28.67±2.90 RFU/μm² at high NP concentration, 1.29±0.01 RFU/μm² at low NP concentration) (FIG. 3 D, E). In addition, it was again noticed that HEPES-NaCl not only provided the densest particle coverage, but preserved replicability in coating density, defined by a low coefficient of variation at high NP concentration (31.97±32.39% in HEPES-NaCl vs. 101.83±152.98% in HEPES and 20.20±14.57% in dH₂O) and at low NP concentration (25.49±13.30% in HEPES-NaCl vs. 140.26±170.84% in HEPES and 1.76±0.88% in dH₂O) between the mean fluorescence intensity of imaged samples (FIGS. 8, 13). Low and high NP concentrations were 0.5 mg/mL (FIG. 3 E panels i-iii) and 2 mg/mL of liposomes in loading buffer (FIG. 3 E panels iv-vi), respectfully. It was visually observed and quantitively confirmed that coating density can be significantly increased with increasing liposome concentration (Table 1). These findings confirm that pH stabilization, ionic strength, and liposome concentration can each influence adsorption outcomes, providing straightforward parameters for adjusting surface coverage.

It was next investigated whether corona poling of the scaffold is required for liposome adsorption, or if optimized solution conditions and mechanical agitation during loading alone are sufficient. In deionized water, uniform liposome adsorption was only observed on corona-poled scaffolds (28.67±2.90 RFU/μm² on corona-poled scaffolds vs. 0.32±0.17 RFU/μm² on unpoled scaffolds, both at high NP concentration) (FIG. 3 D, E iv-vi, vii), indicating that under unbuffered and low-ionic strength conditions, corona poling of the scaffold is required to create sufficient charge for adsorption. However, in HEPES and HEPES-NaCl buffer at high NP concentrations, adsorption occurred efficiently on both poled and unpoled scaffolds, (in HEPES, 18.07=9.20 RFU/μm² on corona-poled scaffolds, 32.79±0.31 RFU/μm² on unpoled scaffolds, and in HEPES NaCl, 19.40±1.24 RFU/μm² on corona-poled scaffolds, 35.54±0.60 RFU/μm² on unpoled scaffolds) (FIG. 3 D, E panels v, viii, vi, ix), suggesting that optimized solution conditions can compensate for the absence of corona poling during vacuum-assisted loading. Further, unpoled scaffolds coated at high NP concentration in HEPES-NaCl had denser particle coatings than the in the other buffers, and relatively low coefficients of variation (3.37±2.39% in HEPES-NaCl vs. 1.88±0.94% in HEPES and 105.62±108.63% in dH2O) (FIG. 8), suggesting that the control of pH and ionic strength when coating unpoled scaffolds leads to more consistent particle coatings.

Further, a morphological difference in coatings using either corona poled or unpoled scaffolds was observed: corona poled scaffolds showed more punctate, clustered fluorescence (FIG. 3E panels iv, vi), while unpoled scaffolds exhibited a more uniform coating (FIG. 3D panels vii, ix). This difference was not observed in HEPES buffer without NaCl, where the coating morphology appeared similarly uniform between poled and unpoled scaffolds (FIG. 3D panels v, viii). It is possible that in the presence of NaCl, the stronger or more spatially defined surface charges on poled scaffolds promote local liposome aggregation during adsorption, as inter-liposomal charge repulsion is reduced due to electrostatic screening. Overall, these findings suggest that transient charge domains generated by mechanical deformation are sufficient to support robust nanoparticle adsorption under optimal buffer conditions, eliminating the need for time-intensive corona poling for a more efficient synthetic pipeline.

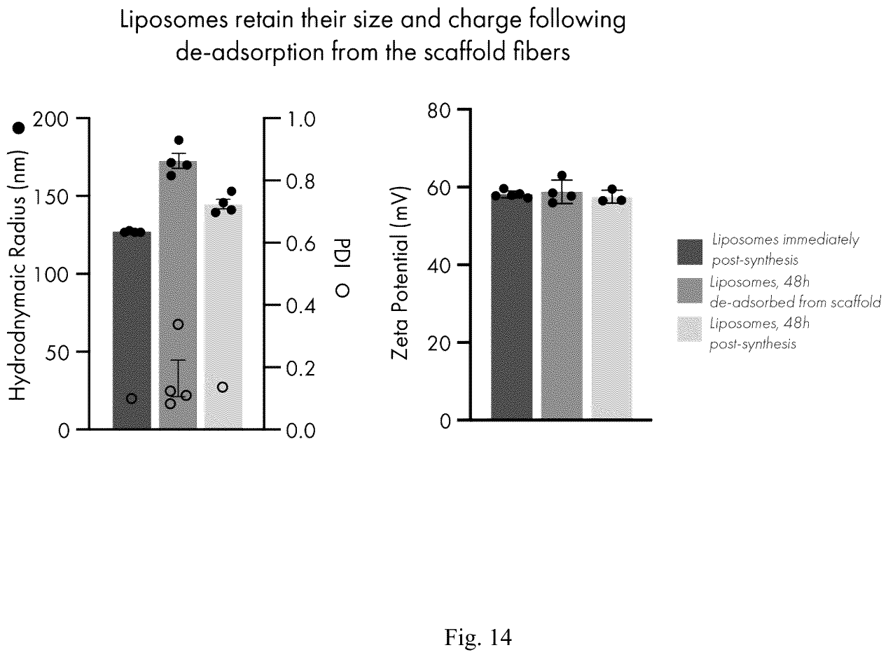

To interrogate whether the morphological difference observed with liposomal coatings was due to the hydrophobic interaction-driven organization of the lipid particles enabling fusion or entropic rearrangement into a supported lipid bilayer, the hydrodynamic radius and zeta potential of the particles was characterized immediately post-synthesis and after de-adsorption from the scaffold. It was observed that particles released from the scaffold had a similar size, polydispersity index, and zeta potential to when they were initially loaded on the scaffold (172.55±4.80 nm, PDI=0.16±0.06 compared to 128.4±2.06 nm, PDI=0.099±0.02 immediately post-synthesis and 58.81±1.51 mV compared to 58.15±0.41 mV immediately post-synthesis) (FIG. 14). The slight increase in size is consistent with reported size changes in liposomes stored in aqueous solutions and is likely not indicative of liposome fusion or particle instability.⁸⁵ This is further supported by the low polydispersity index, indicating a uniform and monodisperse population.

While liposomes adsorbed efficiently to PVDF-TrFE scaffolds under optimized conditions, several open questions remain. Corona poling was still necessary in low-ionic strength buffers and altered liposome coating morphology in the presence of salt, suggesting that scaffold polarization state may influence charge distribution and nanoparticle organization. The underlying mechanism remains unclear and could involve particle aggregation or localized differences in surface potential. Further, a difference in how much nanoparticle was needed to saturate the scaffold using liposomes compared to polymeric nanoparticles was observed. It was hypothesized that the difference in charge density of the nanoparticle types could explain the apparent dose-response using liposomes that was not observed with CMLs, which saturate the scaffold even at low nanoparticle concentrations. This saturation mimics a layer-by-layer materials assembly technique, which employs an excess of material to saturate a surface. A reduction in the scaffold's piezoelectric coefficient following liposome coating was observed. Although this is consistent with surface-level dielectric effects reported in other soft materials,^{84,85} it is not yet known how this change affects scaffold performance in biological contexts. Cells may engage with the scaffold by adhering, deforming fibers, or internalizing surface-bound materials, all of which could dynamically alter charge distribution or local piezoelectric behavior. Future studies will be needed to assess how surface coatings influence bioelectrical signaling during cell-material interactions.

FIGS. 3A-3E depict electrostatic liposome adsorption onto PVDF-TrFE scaffolds. FIG. 3A depicts hydrodynamic radius and zeta potential of liposomes via dynamic light scattering with n=3 replicates. FIG. 3B depicts energy-dispersive X-ray spectroscopy measurements of nitrogen levels on high NP-concentration (2 mg/mL) coated vs. uncoated scaffolds. FIG. 3C depicts piezoelectric coefficient, d33, on 2 mg/mL liposome-coated and uncoated scaffolds, pre- and post-ethanol wash. FIG. 3D depicts image-based quantification of loading using images in FIG. 3E fluorescence microscopy of Cy5 labeled liposomes on scaffolds coated in deionized water, HEPES, and HEPES-NaCl (top to bottom) at low NP concentration (0.5 mg/mL) or high NP concentration (2 mg/mL) on corona-poled scaffolds, or high NP concentration on a non-corona poled scaffold (left to right); scale bar=100 μm and is applied to all groups. Images are collected at the same laser power and are contrast-matched using Fiji-ImageJ. All values are represented as mean±SEM and pair-wise statistics are performed using student's paired t-tests. Multiple comparisons following two-way ANOVA for image-based fluorescence quantification and d33 measurements are corrected by Tukey's multiple comparisons test.

### Example 3—Biocompatibility and Immune Cell Engagement with Liposome-Coated Scaffolds

Annealed PVDF-TrFE is a synthetic, non-degradable polymer that is intrinsically hydrophobic, with a reported water contact angle of 134.3±4.3°.⁴⁶ While these properties make it mechanically and chemically stable for long-term use, they can also limit spontaneous cell-material interactions and necessitate careful evaluation of cytocompatibility following surface modification. Unmodified PVDF-TrFE scaffolds have been widely used for tissue engineering,^{30,88-90} but the introduction of cationic nanoparticle coatings introduces questions of coating stability and new interfaces that may influence both cell viability and behavior.

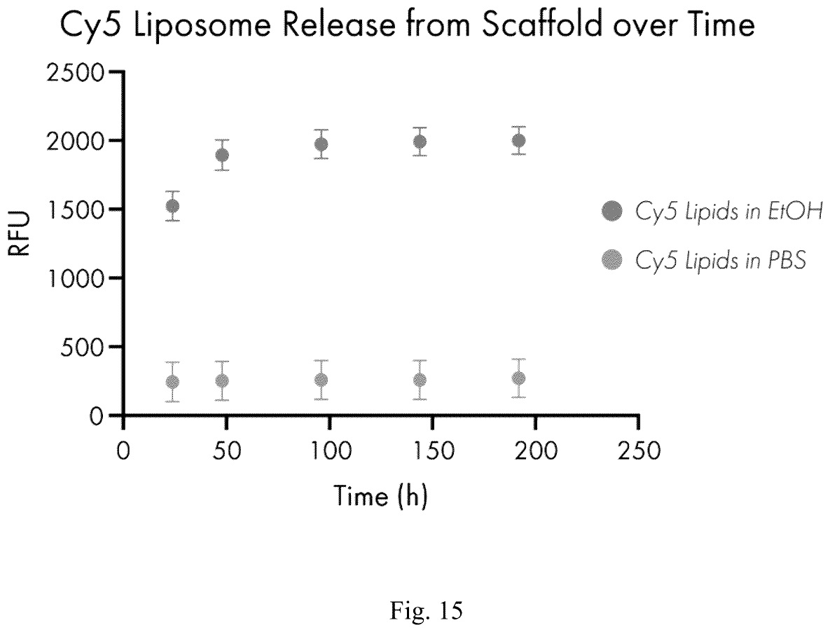

To assess the stability of liposome coatings under aqueous conditions relevant to cell culture, the release of fluorescent lipids from fluorescent liposome-coated scaffolds over time was quantified. Lipid release in aqueous solution (phosphate-buffered saline, PBS) was compared to release in ethanol, which served as positive control for complete particle solubilization. While the majority of fluorescent lipids were rapidly released into ethanol within 24 hours, with near-complete release by 48 hours, minimal lipid release was detected in PBS for up to 192 hours of incubation, indicating sustained retention of liposomes on the scaffold under physiologically relevant aqueous conditions (FIG. 15).

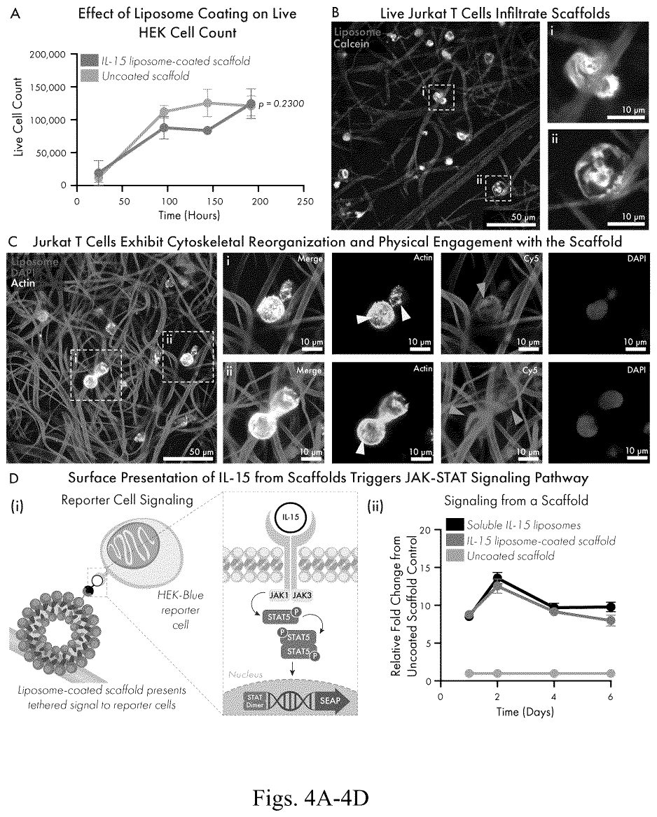

Cytocompatibility and cell engagement of liposome-coated scaffolds using both adherent and suspension cell models was assessed: HEK293 cells were used as a standard model for assessing general cytocompatibility, and Jurkat T cells were used to explore the potential of this platform for ex-vivo immune cell culture and applications within in situ cell therapy manufacturing. HEK293 cells were cultured on uncoated or cationic liposome-coated PVDF-TrFE scaffolds for eight days in low-adherence culture plates to promote scaffold seeding. Cell viability was quantified using the CCK8 assay, which measures metabolic activity via WST-8 reduction. HEK293 cells remained viable throughout the culture period, with no significant difference in metabolic activity between coated and uncoated scaffolds (p=0.2300; FIG. 4A). Jurkat T cells also remained viable when cultured on liposome-coated scaffolds, as confirmed by Calcein AM staining after three days in culture (FIG. 4B).

To evaluate cell-material engagement, cytoskeletal organization was examined and it was assessed whether components of the liposome coating were transferred to the cell membrane. Jurkat T cells seeded onto Cy5-labeled liposome-coated scaffolds were fixed and stained for F-actin and DAPI on day four of cell culture. Confocal microscopy revealed that cells were adhered to scaffold fibers and exhibited a cortical actin distribution, with occasional actin-rich puncta at points of fiber contact (FIG. 3C). In addition, Cy5-labeled lipids from the scaffold coating were visible within the plasma membranes of Jurkat T cells (FIG. 4C, panels i,ii). This membrane-associated signal suggests that cells actively engage with and take up components of the scaffold's liposome coating.

These findings indicate that adding a cationic liposome coating preserves this biocompatibility while introducing a means for tunable nanomaterial presentation. Jurkat T cell behavior on these scaffolds was consistent with typical behaviors of immune cells interacting with three-dimensional substrates,⁸⁹ despite the inherently hydrophobic nature of the PVDF-TrFE. Moreover, the incorporation of Cy5-labeled lipids into the cell membrane indicates active transfer of nanomaterials from the scaffold to cells, potentially through lipid exchange or membrane fusion 90,91

FIGS. 4A-4D depict liposome-coated scaffolds are suitable substrates for cells and enable cytokine presentation. FIG. 4A shows that live HEK293 cell counts were measured from multiple time points on scaffolds with and without cationic liposome coating. Data is presented as mean±SEM from 3 independent wells at each time point. Statistical significance was assessed by two-way ANOVA with Sidak's multiple comparisons test, comparing coated and uncoating scaffold at each time point. P values reflect differences between groups at individual time points, and all error bars are present on graph although some are too small to see. FIG. 4B shows that scaffolds are coated with fluorescent Cy5-labeled liposomes and are imaged with confocal microscopy. Jurkat T cells are stained with Calcein cultured on Cy5 liposome-coated scaffolds and imaged at 60× resolution 3 days after plating. FIG. 4C shows that Jurkat T cells are fixed on day 4 and stained intracellularly with ActinProbe555 and DAPI. Arrows indicate punctate actin (yellow) or liposome uptake (magenta). Scale bars are 50 μm or 10 μm. FIG. 4D shows activating the JAK-STAT pathway with IL-15 liposomes on scaffolds: panel (i) scaffolds are coated with IL-15-tethered liposomes in optimized HEPES-NaCl buffer conditions and SEAP is produced in response to IL-15 recognition. Panel (ii) IL-15 reporter cells are plated on scaffolds, and absorbance is read daily, correlating to the downstream SEAP production from activation of the JAK-STAT pathway with n=3 samples per group. Data is presented as relative fold change from uncoated scaffold control, with mean±SD. Significance is conducted with an Ordinary Two-Way Anova with multiple comparisons using Sadik's multiple comparisons test.

### Example 4—Surface Presentation of IL-15 Cytokine from Scaffolds Supports Functional JAK-STAT Signaling

Having established that liposome-coated scaffolds support cell viability, it was next investigated whether the scaffold interface could locally present functional signaling proteins in a surface-bound configuration. In contrast to soluble cytokine delivery, which is often limited by the cytokine's rapid diffusion, degradation, and off-target effects, surface-tethered presentation enables more stable and spatially confined signal delivery.⁹² Histidine-nickel affinity interactions were previously used to tether recombinant cytokines to liposome surfaces in hydrogel systems,⁷⁹ and it was hypothesized that this supramolecular approach could be extended to liposomes adsorbed onto PVDF-TrFE scaffolds. To test this, this study focused on interleukin-15 (IL-15), a cytokine of interest for ex-vivo immune cell culture⁹³ due to its role in promoting the survival and expansion of memory CD8+ T cells and NK cells.^{94,95,10}

To tether IL-15 to the scaffold, 0.3 mol % 79 of the nickel-chelating lipid DGS-NTA(Ni) was incorporated into the liposome bilayer and adsorbed the functionalized liposomes onto PVDF-TrFE scaffolds using the optimized vacuum-assisted loading protocol with HEPES-NaCl loading buffer. HIS-tagged recombinant IL-15 was bound to the liposomes via overnight incubation prior to coating liposomes onto scaffolds. To assess IL-15 bioactivity, IL-15 and IL-2 reporter HEK293 reporter cells (HEK-Blue CD122/132 reporter cells were used, which secrete alkaline phosphatase (SEAP) in response to IL-15 receptor mediated phosphorylation of STAT5 (FIG. 4D panel i). Reporter cells were seeded directly onto scaffolds in a non-TC-treated plate to encourage cell/scaffold integration, and SEAP levels were independently measured over the course of six days. The following were compared IL-15 liposome-functionalized scaffolds, uncoated scaffolds with soluble IL-15, and uncoated scaffolds without cytokine. STAT-5 phosphorylation kinetics increased similarly between cells cultured on scaffolds with soluble IL-15 as on scaffolds presenting liposome-bound IL-15 for days 1, 2, and 4 (p=0.9184, p=0.2453, p=0.0167, respectively) and only a moderate decrease in intensity was noted on day 6 (79.47% relative to soluble, p=0.0014) (FIG. 4D panel ii). This reduction likely reflects minor IL-15 loss during coating, as the soluble control corresponded to 100% theoretical scaffold loading (0.477 μM IL-15).

These results confirm that scaffold-tethered IL-15 remains bioactive and capable of engaging cell-surface receptors. More broadly, they demonstrate that liposome-coated scaffolds provide a modular platform for spatially confined cytokine presentation. By displaying IL-15 at the scaffold surface through supramolecular tethering, cytokine function was preserved and localized receptor-mediated signaling was achieved without requiring covalent modification of either the cytokine or the scaffold. This flexible strategy enables the facile incorporation of HIS-tagged proteins into electrospun materials and is valuable for ex-vivo immune cell expansion and reprogramming.

This work establishes a modular strategy for post-fabrication functionalization of piezoelectric electrospun scaffolds using electrostatically adsorbed nanocarriers. By combining vacuum-assisted loading with buffer-optimized solution conditions, stable adsorption of charged nanoparticles, including both polymeric and lipid nanoparticles, onto PVDF-TrFE scaffolds was enabled. This modular platform was applied to a biomedically relevant context by generating scaffolds coated with liposomes decorated with immunomodulatory cytokines. These nanocoated scaffolds supported cell viability, enabled lipid exchange between immune cells and scaffold-bound liposomes, and activated cellular signaling pathways. Together, these findings demonstrate that the platform supports localized cytokine presentation and highlight its use as an immune-instructive biomaterial in applications such as ex-vivo 3D cell culture.

This electrostatic coating strategy can be used for a broad range of surface-charged nanocarriers, including extracellular vesicles, polymeric micelles, hybrid nanoparticles, and solid lipid nanoparticles (LNPs). Incorporating LNPs would enable scaffold-tethered gene delivery, combining nucleic acid cargo with the structural and biochemical advantages of electrospun materials. The use of liposomes as a modular display vehicle also allows for multivalent or combinatorial presentation of proteins, such as co-delivered cytokines, chemokines, and growth factors. More broadly, the ability to tune surface coverage through buffer composition and nanoparticle concentration offers a simple and scalable method for customizing scaffold interfaces. Together, these features position this approach as a flexible foundation for engineering multifunctional, cell-responsive scaffolds with potential applications in regenerative medicine, immune engineering, and ex-vivo cell manufacturing.

### Supporting Methods

### PVDF-TrFE Scaffold Fabrication

Scaffolds are electrospun by modifying previously reported methods.^{47,86} Briefly, 20% (w/v) solutions of Polyvinylidene fluoride tri-fluoroethylene (PVDF-TrFE, 70/30) (400 kDa, polydispersity index (PDI) of 2.1, Solvay Solexis Inc., Thorofare, NJ) in 2-Butanone/methyl ethyl ketone (Sigma-Aldrich #360473) are filled into a 10 ml syringe and extruded though a 18-gauge needle, with a 3 ml/hour flow rate. A voltage of 22 kV is connected to the needle tip and randomly oriented fibers are collected on a grounded metal collector plate placed 22-24 cm apart from the needle tip. The fibers are spun at room temperature (~20 °C) with 20-30% humidity followed by aerating the electrospun fibrous mat for at least 48 hours. The scaffolds are annealed at 135 °C for 96 hours and quenched as described in previous methods.³⁰

Corona poling: The scaffolds are Corona poled using a custom setup described previously.⁴⁷ Briefly, the scaffolds are heated to a temperature of ~47 °C for 3 hours on a grounded heating plate. The positive charge is deposited on the scaffold surface using needle tips connected to a voltage of 14 KV. A metal grid with 2 KV voltage is set 2 cm above the scaffolds to distribute the positive charges.

Scaffold fiber diameter measurements: A total of 40 fibers from different spots of 5 distinct SEM images were quantified using Fiji-Image J.

### Layer-by-Layer Nanoparticle Synthesis

100 nm Carboxyl-modified latex nanoparticles (CML) (ThermoFisher Scientific #F8801) are coated in three layers of poly-allylamine hydrochloride (PAH) (Sigma-Aldrich #283215), poly(acrylic acid, sodium salt) solution (PAA) (Sigma-Aldrich #416029) and PAH. PAH is prepared at 4 mg/mL and 6 mg/mL in MilliQ water. PAA is prepared at 4 mg/mL in MilliQ water. CML are prepared at 1 mg/mL and are combined with equal volume of PAH. The solution is pipetted in a bath sonicator three times. The particles are spun at 25,830×g for 45 minutes at 4 °C. The supernatant is removed, and 0.8 times the original particle volume minus the particle volume remaining of MilliQ water is added to the solution to resuspend the particles. Equal volume of 4 mg/mL PAA is added to the particle solution by pipetting inside a bath sonicator. The particles are spun at 25,830×g for 45 minutes at 4 °C. The supernatant is again removed, and 0.8 times the original particle volume minus the particle volume remaining of MilliQ water is added to the solution to resuspend the particles. Equal volume of 6 mg/mL PAA is added to the particle solution by pipetting inside a bath sonicator. The particles are spun at 25,830×g for 45 minutes at 4 °C. The supernatant is removed, and the particles are resuspended in loading buffer.

### Liposome Synthesis

Liposomes are prepared using the thin-film rehydration technique: 1,2-distearoyl-sn-glycero-3-phosphocholine (18:0 PC (DSPC)) (Avanti Polar Lipids #850365), 1,2-dioleoyl-3-trimethylammonium-propane (chloride salt) (18:1 TAP (DOTAP)), (Avanti Polar Lipids #890890), cholesterol (plant) (Avanti Polar Lipids #700100), and 1,2-dioleoyl-sn-glycero-3-[(N-(5-amino-1-carboxypentyl)iminodiacetic acid) succinyl] (nickel salt) (18:1 DGS-NTA(Ni)) (Avanti Polar Lipids #790404) are dissolved at a 9:1:2:0.379 molar ratio in chloroform. Solvent is removed by evaporation under vacuum using a rotovap with bath at 65 °C. The thin film is rehydrated at the desired concentration in loading buffer by in a bath sonicator at 60 °C for 30 mins. For fluorescent liposomes only, 1,2-distearoyl-sn-glycero-3-phosphoethanolamine-N-(Cyanine 5) fluorescent lipid (18:0 Cy5 PE) (Avanti Polar Lipids #810345) is added at 0.01 mol % during sonication. Liposomes are extruded at 60 °C (Avanti Research #610000) using 10 mm filter supports (Avanti Polar Lipids #610014) through PC 1.0 μm membrane (Avanti Research #610010), PC 0.4 μm membrane (Avanti Research #610007), PC 0.2 μm membrane (Avanti Research #610006) and PC 0.1 μm membrane (Avanti Research #610005).

### Nanoparticle Characterization

Nanoparticle hydrodynamic radius and polydispersity index were measured using dynamic light scattering (Malvern Zetasizer Advanced Pro Red, 2=633 nm, dispersant RI=1.33. Zeta potential measurements were also acquired with the Malvern Zetasizer, using laser Doppler electrophoresis. Nanoparticles were diluted 1:1000 in Milli-Q water, with no added salts, in polystyrene Greiner Bio-One Semi-micro/Macro cuvettes (Fisher Scientific #07-000-571) or Folded Capillary Zeta Cell (Malvern Paranalytical #DTS1070) for characterization.

### Scaffold Loading

Loading buffers were formulated in MilliQ water: HEPES-NaCl (500 mM HEPES 400 mM NaCl) and 400 mM NaCl; HEPES Sodium Salt (White Powder), Fisher BioReagents (Fisher Scientific #BP410-500), Sodium Chloride (Fisher Scientific #BP358-1). For all experiments, the scaffold had a size of 6 mm diameter×0.35 mm thickness, which were cut from the electrospun mat using a biopsy punch (Integra #33-36). For scaffold loading by incubation, the scaffold disks were placed in a round-bottom 96-well plate. 200 μL of nanoparticle solution at the desired concentration was added and scaffolds were incubated for 1 hour, after which they were washed three times with MilliQ water. For vacuum-assisted loading, 6 mm scaffold disks were transferred into separate 5 ml polypropylene round-bottom tubes (Corning #352063). 265 μL of the nanoparticle mixture at the desired concentration in loading buffer was added to each tube and capped. A 16-gauge needle (BD #305195) attached to a 50 ml syringe (BD #309654) was inserted into the cap of the tube. The plunger was pulled back to the 20 mL mark to create a vacuum, and dragged across the uneven surface of a 1.5 mL 80-well microtube rack to agitate it three times. This process was performed twice. The tubes were then incubated for one hour at 4 °C, after which the mixture was aspirated, and the scaffolds were rinsed three times with deionized water. All scaffolds used in these experiments were from the same batch of annealed electrospun scaffold mat.

Characterization of Particles De-Adsorbed from Scaffolds

Particles were coated on scaffolds as previously described and incubated in 2 mL Eppendorf tubes in 1 mL of 1×PBS at 4 °C on a spinning rotary shaker. After 48 h, the PBS was collected and characterized with dynamic light scattering as previously described.

Separately, scaffolds were coated with liposomes and incubated on a rotary shaker at 4 °C in 1 mL of either 1×PBS or 70% ethanol. Solution was collected and replaced at each timepoint. Fluorescence of solution was measured at endpoint using a Qubit 4 Fluorometer (Invivogen), exciting with the red 635 nm laser for Cy5 liposomes.

### Scaffold Imaging

The CML and LbL CML nanoparticle-loaded scaffolds were air dried overnight prior to SEM sample preparation. The samples were 8-10 nm sputter-coated with gold-palladium (Cressington sputter coater-108 auto) and imaged using Zeiss Sigma VP SEM with an accelerating voltage of 5 KV. At least n=2 images were captured on two different areas from each scaffold with approximately 1500× and 7000× magnification.

Fluorescent liposome-coated scaffolds and fluorescent Fluosphere (fluorescent CML)-coated scaffolds (Invivogen #F8801) were imaged using the Nikon Ti2 inverted microscope with AXR resonant spectral scanning confocal unit with laser emission range 662 nm-737 nm. Scaffolds were prepared on a microscope slide and imaged through a coverslip. At least n=2 scaffolds were imaged per condition. 50 μm z-stacks were acquired to create maximum intensity projections. Images were denoised by the Nikon software and processed using FIJI-ImageJ. Fluorescence was then quantified with FIJI-ImageJ software: briefly, flattened images were converted to 16-bit images, background was subtracted with a rolling ball radius of 50.0 pixels, and fluorescence was measured. Reported values are intensity density per unit area, or mean intensity density.

### Elemental Analysis

Energy dispersive spectroscopy (EDS) was used for quantitative analysis of liposome-coating. Liposome-coated scaffolds were dried for 48 hours prior to analysis. Samples were coated with 10 nm carbon and imaged using the Screening Cube II (SRAMM). EDS measurements were collected with 20,000 counts at approximately 8100× magnification. n=2 samples were prepared for each group, and an unpaired student's t-test was performed.

Piezoelectric Coefficient (d33) Measurements

Annealed scaffolds were cut into 12 mm diameter×0.35 mm thickness disks, and the piezoelectric coefficient was measured with a piezometer (Piezotest-PM300). The scaffolds were then coated with liposomes using our vacuum-assisted loading technique and airdried overnight. No samples were measured more than twice, and all values were validated with a positive and negative control.

### Cell Culture

HEK Blue™ CD122/CD132 (Invivogen #hkb-il2bg) cells were maintained in Dulbecco's Modified Eagle's Medium (DMEM) (Corning #10-014-CM) supplemented with 10% heat-inactivated fetal bovine serum (Fisher Scientific #A5256701), 100 U/mL penicillin, 100 μg/mL streptomycin (Thermo Fisher Scientific #15140122) 2 mM L-Glutamine (Millipore Sigma #G7513), 100 μg/mL Normocin™ (Invivogen #ant-nr), supplemented with 1×HEK-Blue™ Selection (Invivogen #hb-sel) and 1 μg/mL Puromycin (Invivogen #ant-pr) to select for reporter cells. Cells are tested for Mycoplasma and are maintained at 37 °C and 5% CO2.

### Cell Viability

Cationic liposomes were formulated in sterile HEPES-NaCl (500 mM HEPES 400 mM NaCl) buffer. Scaffolds were loaded with liposomes or sterile buffer via vacuum-assisted loading for each time point. HEK Blue™ CD122/CD132 (Invivogen #hkb-il2bg) cells were plated on scaffolds, and this procedure was repeated every other day, in addition to replacing the cell media in each scaffold well, to enable a comparable readout at the end timepoint. Eight days after plating the first timepoint, Apexbio Technology LLC Cell Counting Kit-8 (CCK-8) reagent (Fisher Scientific #50-190-5565) was added to the scaffold-cell solution achieve a final concentration of 10% CCK-8 reagent in the cell culture well. A standard curve was generated to interpolate cell count values using simple linear regression. The plate was agitated and incubated for three hours at 37 °C, at which point the plate was agitated again in the Synergy Neo2 Plate reader, scaffolds were removed, and endpoint absorbance was read at 450 nm.

### Cell Signaling Assay

Scaffolds are punched using 6 mm biopsy punches (Integra Life Sciences #33-36) and UV-treated for 15 mins on each side. Liposomes are incubated with recombinant Histidine-tagged human IL-15 (Sino Biological #10360-H07E) overnight at 4 °C to achieve a concentration of 180 ng/ml IL-15 on liposomes at 2 mg/mL, the loading concentration, in HEPES-NaCl. Scaffolds are coated with IL-15 liposomes as previously described with sterile vacuum-assisted loading techniques and are placed in flat-bottom propylene non-TC treated 96-well plates (Fisher Scientific cat #07-200-696). Scaffolds are incubated with complete media overnight at 4 °C. Media is removed, and 50,000 HEK-Blue CD122/CD132 (Invivogen #hkb-il2bg) cells at passage 8 are added to the liposome-coated scaffolds, uncoated scaffolds, or soluble condition (containing 0.477 μM of IL-15, the amount of 100% loading efficiency of IL-15 on scaffolds) in 100 μL complete media without Normocin or selective antibiotics. Every 24 hours, the media is collected to be stored at 4 °C. One week after plating, QUANTI-Blue™ solution (Invivogen #rep-qbs) is prepared and incubated at a 10:1 ratio with collected media for 30 minutes at 37 °C. SEAP levels are obtained with endpoint absorbance readings using the Synergy Neo2 plate reader at 620 nm.

### ADDITIONAL FIGURES AND TABLE

FIG. 5 depicts nanoparticle summary data. Dynamic light scattering is used to characterize nanoparticle size, polydispersity index, and zeta potential. Carboxylate modified latex (CML) particles (top), layer-by-layer CML (LbL CML) (middle), and liposomes (bottom) are shown. Results are reported as the mean of n=3 independent syntheses. Individual mean data points are plotted blow, and results are represented as mean±SEM.

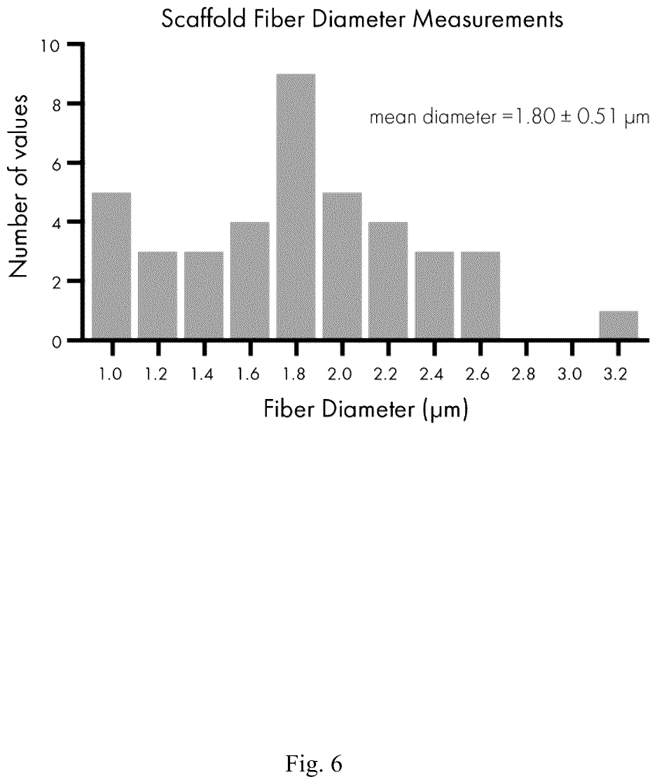

FIG. 6 depicts scaffold fiber size distribution. 40 fiber measurements are collected using Fiji-ImageJ from 5 independent scanning electron microscopy images, and a histogram of the frequency distribution is shown. Summary result of fiber diameter is reported as mean±SD.

FIG. 7 depicts anionic CML coating heterogeneity. Confocal microscopy of multiple independent scaffolds coated with fluorescent anionic CML in dH2O (top), HEPES (middle), or HEPES-NaCl at low (0.5 mg/mL) and high (2 mg/mL) nanoparticle concentrations. N=4 maximum intensity projections of 50 μm stacks were collected per condition, and the scale bar=100 μm.

FIG. 8 depicts coefficients of variance between mean fluorescence intensity of coated scaffolds in different buffer conditions. Coefficients of variation by buffer type: dH2O (grey), HEPES (blue), and HEPES-NaCl (red). Error bars represent propagated uncertainty.

FIGS. 9A-9B depict nanoparticle dose response for scaffold coating. Scaffolds are vacuum loaded with anionic CML nanoparticles. (A) representative confocal images of maximum intensity projections of 50 μm Z-stacks of nanoparticles at 0.01, 0.1, 0.5, or 2 mg/mL nanoparticle (left to right) loaded in dH2O, HEPES, or HEPES-NaCl buffer (top to bottom). (B) Dose response curve from Fiji-ImageJ quantification of four technical replicate scaffolds from conditions in (A). Scaffolds are coated at varying doses in dH2O (grey), HEPES (blue), or HEPES-NaCl (red). Points are represented as mean±SEM and data is fitted with nonlinear least squares fit.

FIG. 10 depicts uncropped SEM of polymeric nanoparticles on corona poled scaffolds (images from FIG. 2B), collected at 5-10K× magnification.

FIG. 11 depicts low-magnification SEM image of uncoated scaffold (left) compared to scaffold coated in 2 mg/mL LbL CML with HEPES-NaCl (right), images acquired at 1-1.5K× magnification.

FIG. 12 depicts piezoelectric coefficient decreases with all particle coatings. N=3 independent scaffolds are measured. Buffer-loaded scaffold is vacuum-loaded with HEPES-NaCl, and coated scaffolds are loaded with 2 mg/mL of the corresponding particle type in HEPES-NaCl. Values are represented as mean±SEM. Multiple comparisons following ANOVA are corrected with Šídák's multiple comparisons test.

FIG. 13 depicts cationic liposome coating heterogeneity. Confocal microscopy of multiple scaffolds coated with fluorescent cationic liposomes in dH2O (top), HEPES (middle), or HEPES-NaCl using corona-poled scaffolds at low (0.5 mg/mL) density (left) and high (2 mg/mL) density (middle), or unpoled scaffolds at high density (right) nanoparticle concentrations. N=2 maximum intensity projections of 50 μm stacks are shown per condition, and the scale bar=100 μm.

FIG. 14 depicts liposome characterization after de-adsorption. Dynamic light scattering measurements of hydrodynamic radius (left) and zeta potential (right) of liposomes immediately post-synthesis (dark), 48 h de-adsorbed from the scaffold (middle), or 48-hours post-synthesis (light). At least n=3 technical replicates were collected per sample condition. Polydispersity index is shown for hydrodynamic radius measurements, denoted with an open circle.

FIG. 15 shows how particles are retained on scaffolds in aqueous environments. Fluorometric measurements of cumulative fluorescent lipids released from scaffolds into solution over time. Subsequent data points are summations of previous release. 2 mg/mL Cy5-liposome-coated scaffolds prepared in HEPES-NaCl are incubated in 1 mL of either ethanol (dark) or PBS (light) and are shaken on a rotary shaker at 4 °C n=3 technical replicates are measured, and values are represented as mean±SEM.

TABLE 1

Fluorescent CML Imaging Analysis Statistics Summary (FIG. 2 A ii)

| Šídák's | | | | | | | | | | |
|---|---|---|---|---|---|---|---|---|---|---|
| multiple | | | Predicted | | | | | | | |
| comparisons | Predicted | Predicted | (LS) | | | | | | | |
| test, | (LS) | (LS) | mean | SE of | | | | | Sum- | Adjusted |
| a = 0.05 | mean 1 | mean 2 | diff. | diff. | N1 | N2 | t | DF | mary | P Value |
| 0.5 mg/mL | 2.601 | 34.33 | −31.73 | 5.952 | 4 | 4 | 5.33 | 19 | *** | 0.0006 |
| diH2O vs. | | | | | | | | | | |
| 0.5 mg/mL | | | | | | | | | | |
| HEPES | | | | | | | | | | |
| 0.5 mg/mL | 2.601 | 38.76 | −36.16 | 5.952 | 4 | 4 | 6.075 | 19 | *** | 0.0001 |
| diH2O vs. | | | | | | | | | | |
| 0.5 mg/mL | | | | | | | | | | |
| HEPES-NaCl | | | | | | | | | | |
| 0.5 mg/mL | 2.601 | 0.7428 | 1.858 | 5.952 | 4 | 4 | 0.3122 | 19 | ns | >0.9999 |
| diH2O vs. | | | | | | | | | | |
| 2 mg/mL | | | | | | | | | | |
| diH2O | | | | | | | | | | |
| 0.5 mg/mL | 2.601 | 35.19 | −32.59 | 5.952 | 4 | 4 | 5.476 | 19 | *** | 0.0004 |
| diH2O vs. | | | | | | | | | | |
| 2 mg/mL | | | | | | | | | | |
| HEPES | | | | | | | | | | |
| 0.5 mg/mL | 2.601 | 33.12 | −30.52 | 5.646 | 4 | 5 | 5.405 | 19 | *** | 0.0005 |
| diH2O vs. | | | | | | | | | | |
| 2 mg/mL | | | | | | | | | | |
| HEPES-NaCl | | | | | | | | | | |
| 0.5 mg/mL | 34.33 | 38.76 | −4.43 | 5.952 | 4 | 4 | 0.7443 | 19 | ns | >0.9999 |
| HEPES vs. | | | | | | | | | | |
| 0.5 mg/mL | | | | | | | | | | |
| HEPES-NaCl | | | | | | | | | | |
| 0.5 mg/mL | 34.33 | 0.7428 | 33.58 | 5.952 | 4 | 4 | 5.643 | 19 | *** | 0.0003 |
| HEPES vs. | | | | | | | | | | |
| 2 mg/mL | | | | | | | | | | |
| diH2O | | | | | | | | | | |
| 0.5 mg/mL | 34.33 | 35.19 | −0.866 | 5.952 | 4 | 4 | 0.1455 | 19 | ns | >0.9999 |
| HEPES vs. | | | | | | | | | | |
| 2 mg/mL | | | | | | | | | | |
| HEPES | | | | | | | | | | |
| 0.5 mg/mL | 34.33 | 33.12 | 1.206 | 5.646 | 4 | 5 | 0.2136 | 19 | ns | >0.9999 |
| HEPES vs. | | | | | | | | | | |
| 2 mg/mL | | | | | | | | | | |
| HEPES-NaCl | | | | | | | | | | |
| 0.5 mg/mL | 38.76 | 0.7428 | 38.01 | 5.952 | 4 | 4 | 6.387 | 19 | **** | <0.0001 |
| HEPES-NaCl vs. | | | | | | | | | | |
| 2 mg/mL | | | | | | | | | | |
| diH2O | | | | | | | | | | |
| 0.5 mg/mL | 38.76 | 35.19 | 3.564 | 5.952 | 4 | 4 | 0.5988 | 19 | ns | >0.9999 |
| HEPES-NaCl vs. | | | | | | | | | | |
| 2 mg/mL | | | | | | | | | | |
| HEPES | | | | | | | | | | |
| 0.5 mg/mL | 38.76 | 33.12 | 5.636 | 5.646 | 4 | 5 | 0.9982 | 19 | ns | 0.9976 |
| HEPES-NaCl vs. | | | | | | | | | | |
| 2 mg/mL | | | | | | | | | | |
| HEPES-NaCl | | | | | | | | | | |
| 2 mg/mL | 0.7428 | 35.19 | −34.45 | 5.952 | 4 | 4 | 5.788 | 19 | *** | 0.0002 |
| diH2O vs. | | | | | | | | | | |
| 2 mg/mL | | | | | | | | | | |
| HEPES | | | | | | | | | | |
| 2 mg/mL | 0.7428 | 33.12 | −32.38 | 5.646 | 4 | 5 | 5.734 | 19 | *** | 0.0002 |
| diH2O vs. | | | | | | | | | | |
| 2 mg/mL | | | | | | | | | | |
| HEPES-NaCl | | | | | | | | | | |
| 2 mg/mL | 35.19 | 33.12 | 2.072 | 5.646 | 4 | 5 | 0.367 | 19 | ns | >0.9999 |
| HEPES vs. | | | | | | | | | | |
| 2 mg/mL | | | | | | | | | | |
| HEPES-NaCl | | | | | | | | | | |

Fluorescent Liposome Imaging Analysis Statistics Summary (FIG. 3 D)

| Tukey's | | | | | | | | | | |
|---|---|---|---|---|---|---|---|---|---|---|
| multiple | | | Predicted | | | | | | | |
| comparisons | Predicted | Predicted | (LS) | | | | | | | |
| test, | (LS) | (LS) | mean | SE of | | | | | Sum- | Adjusted |
| a = 0.05 | mean 1 | mean 2 | diff. | diff. | N1 | N2 | q | DF | mary | P Value |
| 0.5 mg/mL poled | 1.289 | 8.643 | −7.354 | 6.585 | 2 | 2 | 1.579 | 15 | ns | 0.9623 |
| diH2O vs. | | | | | | | | | | |
| 0.5 mg/mL poled | | | | | | | | | | |
| HEPES | | | | | | | | | | |
| 0.5 mg/mL poled | 1.289 | 10.01 | −8.717 | 6.012 | 2 | 3 | 2.051 | 15 | ns | 0.8613 |
| diH2O vs. | | | | | | | | | | |
| 0.5 mg/mL poled | | | | | | | | | | |
| HEPES-NaCl | | | | | | | | | | |
| 0.5 mg/mL poled | 1.289 | 28.67 | −27.38 | 6.585 | 2 | 2 | 5.88 | 15 | * | 0.0176 |
| diH2O vs. | | | | | | | | | | |
| 2 mg/mL poled | | | | | | | | | | |
| diH2O | | | | | | | | | | |
| 0.5 mg/mL poled | 1.289 | 17.35 | −16.06 | 5.51 | 2 | 5 | 4.122 | 15 | ns | 0.1611 |
| diH2O vs. | | | | | | | | | | |
| 2 mg/mL poled | | | | | | | | | | |
| HEPES | | | | | | | | | | |
| 0.5 mg/mL poled | 1.289 | 23.19 | −21.9 | 6.585 | 2 | 2 | 4.703 | 15 | ns | 0.0801 |
| diH2O vs. | | | | | | | | | | |
| 2 mg/mL poled | | | | | | | | | | |
| HEPES-NaCl | | | | | | | | | | |
| 0.5 mg/mL poled | 1.289 | 0.32 | 0.969 | 6.585 | 2 | 2 | 0.2081 | 15 | ns | >0.9999 |
| diH2O vs. | | | | | | | | | | |
| 2 mg/mL unpoled | | | | | | | | | | |
| diH2O | | | | | | | | | | |
| 0.5 mg/mL poled | 1.289 | 34.16 | −32.87 | 5.703 | 2 | 4 | 8.152 | 15 | *** | 0.0009 |
| diH2O vs. | | | | | | | | | | |
| 2 mg/mL unpoled | | | | | | | | | | |
| HEPES | | | | | | | | | | |
| 0.5 mg/mL poled | 1.289 | 35.54 | −34.25 | 6.585 | 2 | 2 | 7.356 | 15 | ** | 0.0025 |
| diH2O vs. | | | | | | | | | | |
| 2 mg/mL unpoled | | | | | | | | | | |
| HEPES-NaCl | | | | | | | | | | |
| 0.5 mg/mL poled | 8.643 | 10.01 | −1.363 | 6.012 | 2 | 3 | 0.3206 | 15 | ns | >0.9999 |
| HEPES vs. | | | | | | | | | | |
| 0.5 mg/mL poled | | | | | | | | | | |
| HEPES-NaCl | | | | | | | | | | |
| 0.5 mg/mL poled | 8.643 | 28.67 | −20.03 | 6.585 | 2 | 2 | 4.3 | 15 | ns | 0.1308 |
| HEPES vs. | | | | | | | | | | |
| 2 mg/mL poled | | | | | | | | | | |
| diH2O | | | | | | | | | | |
| 0.5 mg/mL poled | 8.643 | 17.35 | −8.704 | 5.51 | 2 | 5 | 2.234 | 15 | ns | 0.8015 |
| HEPES vs. | | | | | | | | | | |
| 2 mg/mL poled | | | | | | | | | | |
| HEPES | | | | | | | | | | |
| 0.5 mg/mL poled | 8.643 | 23.19 | −14.55 | 6.585 | 2 | 2 | 3.124 | 15 | ns | 0.4463 |
| HEPES vs. | | | | | | | | | | |
| 2 mg/mL poled | | | | | | | | | | |
| HEPES-NaCl | | | | | | | | | | |
| 0.5 mg/mL poled | 8.643 | 0.32 | 8.323 | 6.585 | 2 | 2 | 1.787 | 15 | ns | 0.9274 |
| HEPES vs. | | | | | | | | | | |
| 2 mg/mL unpoled | | | | | | | | | | |
| diH2O | | | | | | | | | | |
| 0.5 mg/mL poled | 8.643 | 34.16 | −25.52 | 5.703 | 2 | 4 | 6.328 | 15 | ** | 0.0097 |
| HEPES vs. | | | | | | | | | | |
| 2 mg/mL unpoled | | | | | | | | | | |
| HEPES | | | | | | | | | | |
| 0.5 mg/mL poled | 8.643 | 35.54 | −26.9 | 6.585 | 2 | 2 | 5.776 | 15 | * | 0.0202 |
| HEPES vs. | | | | | | | | | | |
| 2 mg/mL unpoled | | | | | | | | | | |
| HEPES-NaCl | | | | | | | | | | |
| 0.5 mg/mL poled | 10.01 | 28.67 | −18.66 | 6.012 | 3 | 2 | 4.39 | 15 | ns | 0.1175 |
| HEPES-NaCl vs. | | | | | | | | | | |
| 2 mg/mL poled | | | | | | | | | | |
| diH2O | | | | | | | | | | |
| 0.5 mg/mL poled | 10.01 | 17.35 | −7.341 | 4.809 | 3 | 5 | 2.159 | 15 | ns | 0.8272 |
| HEPES-NaCl vs. | | | | | | | | | | |
| 2 mg/mL poled | | | | | | | | | | |
| HEPES | | | | | | | | | | |
| 0.5 mg/mL poled | 10.01 | 23.19 | −13.18 | 6.012 | 3 | 2 | 3.102 | 15 | ns | 0.4549 |
| HEPES-NaCl vs. | | | | | | | | | | |
| 2 mg/mL poled | | | | | | | | | | |
| HEPES-NaCl | | | | | | | | | | |
| 0.5 mg/mL poled | 10.01 | 0.32 | 9.686 | 6.012 | 3 | 2 | 2.279 | 15 | ns | 0.7857 |
| HEPES-NaCl vs. | | | | | | | | | | |
| 2 mg/mL unpoled | | | | | | | | | | |
| diH2O | | | | | | | | | | |
| 0.5 mg/mL poled | 10.01 | 34.16 | −24.16 | 5.03 | 3 | 4 | 6.793 | 15 | ** | 0.0053 |
| HEPES-NaCl vs. | | | | | | | | | | |
| 2 mg/mL unpoled | | | | | | | | | | |
| HEPES | | | | | | | | | | |
| 0.5 mg/mL poled | 10.01 | 35.54 | −25.54 | 6.012 | 3 | 2 | 6.007 | 15 | * | 0.0149 |
| HEPES-NaCl vs. | | | | | | | | | | |
| 2 mg/mL unpoled | | | | | | | | | | |
| HEPES-NaCl | | | | | | | | | | |
| 2 mg/mL poled | 28.67 | 17.35 | 11.32 | 5.51 | 2 | 5 | 2.906 | 15 | ns | 0.5329 |
| diH2O vs. | | | | | | | | | | |
| 2 mg/mL poled | | | | | | | | | | |
| HEPES | | | | | | | | | | |
| 2 mg/mL poled | 28.67 | 23.19 | 5.478 | 6.585 | 2 | 2 | 1.176 | 15 | ns | 0.9937 |
| diH2O vs. | | | | | | | | | | |
| 2 mg/mL poled | | | | | | | | | | |
| HEPES-NaCl | | | | | | | | | | |
| 2 mg/mL poled | 28.67 | 0.32 | 28.35 | 6.585 | 2 | 2 | 6.088 | 15 | * | 0.0134 |
| diH2O vs. | | | | | | | | | | |
| 2 mg/mL unpoled | | | | | | | | | | |
| diH2O | | | | | | | | | | |
| 2 mg/mL poled | 28.67 | 34.16 | −5.495 | 5.703 | 2 | 4 | 1.363 | 15 | ns | 0.984 |
| diH2O vs. | | | | | | | | | | |
| 2 mg/mL unpoled | | | | | | | | | | |
| HEPES | | | | | | | | | | |
| 2 mg/mL poled | 28.67 | 35.54 | −6.872 | 6.585 | 2 | 2 | 1.476 | 15 | ns | 0.9743 |
| diH2O vs. | | | | | | | | | | |
| 2 mg/mL unpoled | | | | | | | | | | |
| HEPES-NaCl | | | | | | | | | | |
| 2 mg/mL poled | 17.35 | 23.19 | −5.844 | 5.51 | 5 | 2 | 1.5 | 15 | ns | 0.9718 |
| HEPES vs. | | | | | | | | | | |
| 2 mg/mL poled | | | | | | | | | | |
| HEPES-NaCl | | | | | | | | | | |
| 2 mg/mL poled | 17.35 | 0.32 | 17.03 | 5.51 | 5 | 2 | 4.37 | 15 | ns | 0.1203 |
| HEPES vs. | | | | | | | | | | |
| 2 mg/mL unpoled | | | | | | | | | | |
| diH2O | | | | | | | | | | |
| 2 mg/mL poled | 17.35 | 34.16 | −16.82 | 4.418 | 5 | 4 | 5.384 | 15 | * | 0.0337 |
| HEPES vs. | | | | | | | | | | |
| 2 mg/mL unpoled | | | | | | | | | | |
| HEPES | | | | | | | | | | |
| 2 mg/mL poled | 17.35 | 35.54 | −18.19 | 5.51 | 5 | 2 | 4.67 | 15 | ns | 0.0835 |
| HEPES vs. | | | | | | | | | | |
| 2 mg/mL unpoled | | | | | | | | | | |
| HEPES-NaCl | | | | | | | | | | |
| 2 mg/mL poled | 23.19 | 0.32 | 22.87 | 6.585 | 2 | 2 | 4.911 | 15 | ns | 0.0617 |
| HEPES-NaCl vs. | | | | | | | | | | |
| 2 mg/mL unpoled | | | | | | | | | | |
| diH2O | | | | | | | | | | |
| 2 mg/mL poled | 23.19 | 34.16 | −10.97 | 5.703 | 2 | 4 | 2.721 | 15 | ns | 0.6095 |
| HEPES-NaCl vs. | | | | | | | | | | |
| 2 mg/mL unpoled | | | | | | | | | | |
| HEPES | | | | | | | | | | |
| 2 mg/mL poled | 23.19 | 35.54 | −12.35 | 6.585 | 2 | 2 | 2.652 | 15 | ns | 0.6381 |
| HEPES-NaCl vs. | | | | | | | | | | |
| 2 mg/mL unpoled | | | | | | | | | | |
| HEPES-NaCl | | | | | | | | | | |
| 2 mg/mL unpoled | 0.32 | 34.16 | −33.84 | 5.703 | 2 | 4 | 8.392 | 15 | *** | 0.0007 |
| diH2O vs. | | | | | | | | | | |
| 2 mg/mL unpoled | | | | | | | | | | |
| HEPES | | | | | | | | | | |
| 2 mg/mL unpoled | 0.32 | 35.54 | −35.22 | 6.585 | 2 | 2 | 7.564 | 15 | ** | 0.0019 |
| diH2O vs. | | | | | | | | | | |
| 2 mg/mL unpoled | | | | | | | | | | |
| HEPES-NaCl | | | | | | | | | | |
| 2 mg/mL unpoled | 34.16 | 35.54 | −1.377 | 5.703 | 4 | 2 | 0.3415 | 15 | ns | >0.9999 |
| HEPES vs. | | | | | | | | | | |
| 2 mg/mL unpoled | | | | | | | | | | |
| HEPES-NaCl | | | | | | | | | | |

Piezoelectric Coefficient Before and After Coating (FIG. 3C)

| Tukey's | | | | | | | | | | |
|---|---|---|---|---|---|---|---|---|---|---|
| multiple | | | | | | | | | | |
| comparisons | | | | | | | | | | |
| test, | | | Mean | SE of | | | | | Sum- | Adjusted |
| a = 0.05 | Mean 1 | Mean 2 | Diff. | diff. | n1 | n2 | q | DF | mary | P Value |
| Uncoated | −0.1700 | 0.000 | −0.1700 | 0.02285 | 3 | 3 | 10.52 | 8 | *** | 0.0003 |
| PVDF-TrFE | | | | | | | | | | |
| Scaffold vs. | | | | | | | | | | |
| Liposome-coated, | | | | | | | | | | |
| it = 0 | | | | | | | | | | |
| Uncoated | −0.1700 | −0.07000 | −0.1000 | 0.02285 | 3 | 3 | 6.189 | 8 | * | 0.0101 |
| PVDF-TrFE | | | | | | | | | | |
| Scaffold vs. | | | | | | | | | | |
| Liposome-coated, | | | | | | | | | | |
| EtOH-washed, | | | | | | | | | | |
| t = 192 h | | | | | | | | | | |
| Uncoated | −0.1700 | −0.1067 | −0.06333 | 0.02285 | 3 | 3 | 3.919 | 8 | ns | 0.0919 |
| PVDF-TrFE | | | | | | | | | | |
| Scaffold vs. | | | | | | | | | | |
| Uncoated | | | | | | | | | | |
| PVDF-TrFE | | | | | | | | | | |
| Scaffold, | | | | | | | | | | |
| EtOH washed | | | | | | | | | | |
| Liposome-coated, | 0.000 | −0.07000 | 0.07000 | 0.02285 | 3 | 3 | 4.332 | 8 | ns | 0.0608 |
| t = 0 vs. | | | | | | | | | | |
| Liposome-coated, | | | | | | | | | | |
| EtOH-washed, | | | | | | | | | | |
| t = 192 h | | | | | | | | | | |
| Liposome-coated, | 0.000 | −0.1067 | 0.1067 | 0.02285 | 3 | 3 | 6.601 | 8 | ** | 0.0070 |
| it = 0 vs. | | | | | | | | | | |
| Uncoated | | | | | | | | | | |
| PVDF-TrFE | | | | | | | | | | |
| Scaffold, | | | | | | | | | | |
| EtOH washed | | | | | | | | | | |
| Liposome-coated, | −0.07000 | −0.1067 | 0.03667 | 0.02285 | 3 | 3 | 2.269 | 8 | ns | 0.4277 |
| ETOH-washed, | | | | | | | | | | |
| t = 192 h vs. | | | | | | | | | | |
| Uncoated | | | | | | | | | | |
| PVDF-TrFE | | | | | | | | | | |
| Scaffold, | | | | | | | | | | |
| EtOH washed | | | | | | | | | | |

Effect of Liposome Coating on Live HEK Cell Count (FIG. 4 A)

| Šídák's | | | | | | | | | | |
|---|---|---|---|---|---|---|---|---|---|---|
| multiple | | | | | | | | | | |
| comparisons | | | | | | | | | | |
| test, | | | Mean | SE of | | | | | Sum- | Adjusted |
| a = 0.05 | Mean 1 | Mean 2 | Diff. | diff. | n1 | n2 | t | DF | mary | P Value |
| Liposome | 19215 | 11940 | 7275 | 22357 | 3 | 3 | 0.3254 | 16 | ns | 0.9960 |
| scaffold vs. | | | | | | | | | | |
| uncoated | | | | | | | | | | |
| scaffold, 24 h | | | | | | | | | | |
| Liposome | 87690 | 112148 | −24458 | 22357 | 3 | 3 | 1.094 | 16 | ns | 0.7461 |
| scaffold vs. | | | | | | | | | | |
| uncoated | | | | | | | | | | |
| scaffold, 96 h | | | | | | | | | | |
| Liposome | 83581 | 125458 | −41877 | 22357 | 3 | 3 | 1.873 | 16 | ns | 0.2819 |
| scaffold vs. | | | | | | | | | | |
| uncoated | | | | | | | | | | |
| scaffold, 144 h | | | | | | | | | | |
| Liposome | 124409 | 121157 | 3253 | 22357 | 3 | 3 | 0.1455 | 16 | ns | 0.9998 |
| scaffold vs. | | | | | | | | | | |
| uncoated | | | | | | | | | | |
| scaffold, 192 h | | | | | | | | | | |

Signaling from a Scaffold (FIG. 4 D ii)

| Šídák's | | | | | | | | | | |
|---|---|---|---|---|---|---|---|---|---|---|
| multiple | | | | | | | | | | |
| comparisons | | | | | | | | | | |
| test, | | | Mean | SE of | | | | | Sum- | Adjusted |
| a = 0.05 | Mean 1 | Mean 2 | Diff. | diff. | n1 | n2 | t | DF | mary | P Value |
| IL-15 scaffold | 0.3817 | 0.3687 | 0.01300 | 0.04921 | 3 | 3 | 0.2642 | 16.00 | ns | 0.9982 |
| vs. uncoated | | | | | | | | | | |
| scaffold, day 1 | | | | | | | | | | |
| IL-15 scaffold | 0.7040 | 0.7687 | −0.06467 | 0.04921 | 3 | 3 | 1.314 | 16.00 | ns | 0.6052 |
| vs. uncoated | | | | | | | | | | |
| scaffold, day 2 | | | | | | | | | | |
| IL-15 scaffold | 0.9357 | 0.9967 | −0.06100 | 0.04921 | 3 | 3 | 1.240 | 16.00 | ns | 0.6539 |
| vs. uncoated | | | | | | | | | | |
| scaffold, day 4 | | | | | | | | | | |
| IL-15 scaffold | 0.8617 | 1.084 | −0.2227 | 0.04921 | 3 | 3 | 4.525 | 16.00 | ns | 0.0014 |
| vs. uncoated | | | | | | | | | | |
| scaffold, | | | | | | | | | | |
| day 6 | | | | | | | | | | |

Piezoelectric Coefficient Decreases with Coating (FIG. 12)

| Šídák's | | | | | | | | | | |
|---|---|---|---|---|---|---|---|---|---|---|
| multiple | | | | | | | | | | |
| comparisons | | | | | | | | | | |
| test, | | | Mean | SE of | | | | | Sum- | Adjusted |
| a = 0.05 | Mean 1 | Mean 2 | Diff. | diff. | n1 | n2 | t | DF | mary | P Value |
| PVDF-TrFE | −0.1700 | −0.1233 | −0.04667 | 0.01856 | 3 | 3 | 2.514 | 8 | ns | 0.1981 |
| scaffold vs. | | | | | | | | | | |
| buffer-loaded | | | | | | | | | | |
| scaffold | | | | | | | | | | |
| PVDF-TrFE | −0.1700 | −0.02667 | −0.1433 | 0.01856 | 3 | 3 | 7.723 | 8 | *** | 0.0003 |
| scaffold vs. | | | | | | | | | | |
| CML-coated | | | | | | | | | | |
| scaffold | | | | | | | | | | |
| PVDF-TrFE | −0.1700 | 0.000 | −0.1700 | 0.01856 | 3 | 3 | 9.160 | 8 | **** | <0.0001 |
| scaffold vs. | | | | | | | | | | |
| Liposome-coated | | | | | | | | | | |
| scaffold | | | | | | | | | | |
| buffer-loaded | −0.1233 | −0.02667 | −0.09667 | 0.01856 | 3 | 3 | 5.209 | 8 | ** | 0.0049 |
| scaffold vs. | | | | | | | | | | |
| CML-coated | | | | | | | | | | |
| scaffold | | | | | | | | | | |
| buffer-loaded | −0.1233 | 0.000 | −0.1233 | 0.01856 | 3 | 3 | 6.645 | 8 | *** | 0.0010 |
| scaffold vs. | | | | | | | | | | |
| Liposome-coated | | | | | | | | | | |
| scaffold | | | | | | | | | | |
| CML-coated | −0.02667 | 0.000 | −0.02667 | 0.01856 | 3 | 3 | 1.437 | 8 | ns | 0.7148 |
| scaffold vs. | | | | | | | | | | |
| Liposome-coated | | | | | | | | | | |
| scaffold | | | | | | | | | | |
| PVDF-TrFE | −0.1700 | −0.1233 | −0.04667 | 0.01856 | 3 | 3 | 2.514 | 8 | ns | 0.1981 |
| scaffold vs. | | | | | | | | | | |
| buffer-loaded | | | | | | | | | | |
| scaffold | | | | | | | | | | |

### REFERENCES

1 Cai, L. et al. Engineered biomaterials for cancer immunotherapy. MedComm (2020) 1, 35-46 (2020).
2. Liu, J. M. H., Zhang, X., Joe, S., Luo, X. & Shea, L. D. Evaluation of biomaterial scaffold delivery of IL-33 as a localized immunomodulatory agent to support cell transplantation in adipose tissue. J Immunol Regen Med 1, 1-12 (2018).
3. Shang, Q. et al. Local scaffold-assisted delivery of immunotherapeutic agents for improved cancer immunotherapy. Adv Drug Deliv Rev 185, 114308 (2022).
4. Wang, H. et al. Biomaterial-based scaffold for in situ chemo-immunotherapy to treat poorly immunogenic tumors. Nat Commun 11, 5696 (2020).
5. Zhong, J. X., Raghavan, P. & Desai, T. A. Harnessing Biomaterials for Immunomodulatory-Driven Tissue Engineering. Regen Eng Transl Med 9, 224-239 (2023).
6 Mujahid, K. et al. Biomaterial-Based Sustained-Release Drug Formulations for Localized Cancer Immunotherapy. ACS Appl Bio Mater 7, 4944-4961 (2024).
7. Messina, J. M. et al. Unveiling Cytokine Charge Disparity as a Potential Mechanism for Immune Regulation. Cytokine Growth Factor Rev 77, 1-14 (2024).
8 Kuribayashi, T. Elimination half-lives of interleukin-6 and cytokine-induced neutrophil chemoattractant-1 synthesized in response to inflammatory stimulation in rats. Lab Anim Res 34, 80-83 (2018).
9. Dholakia, J. et al. Development of Delivery Systems for Local Administration of Cytokines/Cytokine Gene-Directed Therapeutics: Modern Oncologic Implications. Curr Oncol Rep 24, 389-397 (2022).
10. Mishra, A., Sullivan, L. & Caligiuri, M. A. Molecular Pathways: Interleukin-15 Signaling in Health and in Cancer. Clinical Cancer Research 20, 2044-2050 (2014).
11. Torres-Ortega, P. V. et al. Encapsulation of MSCs and GDNF in an Injectable Nanoreinforced Supramolecular Hydrogel for Brain Tissue Engineering. Biomacromolecules 23, 4629-4644 (2022).
12. Massena, C. J. et al. A tractable covalent linker strategy for the production of immunogenic antigen-TLR7/8L bioconjugates. Chem Commun (Camb) 57, 4698-4701 (2021).
13. Monteiro, N., Martins, A., Reis, R. L. & Neves, N. M. Nanoparticle-based bioactive agent release systems for bone and cartilage tissue engineering. Regen Ther 1, 109-118 (2015).
14. Grace, M. J. et al. Site of pegylation and polyethylene glycol molecule size attenuate interferon-alpha antiviral and antiproliferative activities through the JAK/STAT signaling pathway. J Biol Chem 280, 6327-6336 (2005).
15. Tsutsumi, Y. et al. Site-specific chemical modification with polyethylene glycol of recombinant immunotoxin anti-Tac (Fv)-PE38 (LMB-2) improves antitumor activity and reduces animal toxicity and immunogenicity. Proceedings of the National Academy of Sciences 97, 8548-8553 (2000).
16. He, X. et al. Recent Progress of Rational Modified Nanocarriers for Cytosolic Protein Delivery. Pharmaceutics 15, 1610 (2023).
17. Jash, A., Ubeyitogullari, A. & Rizvi, S. S. H. Liposomes for oral delivery of protein and peptide-based therapeutics: challenges, formulation strategies, and advances. J Mater Chem B 9, 4773-4792 (2021).
18. Barberio, A. E. et al. Layer-by-layer interleukin-12 nanoparticles drive a safe and effective response in ovarian tumors. Bioengineering & Translational Medicine 8, e10453 (2023).
19. Xing, H., Hwang, K. & Lu, Y. Recent Developments of Liposomes as Nanocarriers for Theranostic Applications. Theranostics 6, 1336-1352 (2016).
20. Frick, M., Schwieger, C. & Schmidt, C. Liposomes as Carriers of Membrane-Associated Proteins and Peptides for Mass Spectrometric Analysis. Angew Chem Int Ed Engl 60, 11523-11530 (2021).
21. Monteiro, N., Martins, A., Reis, R. L. & Neves, N. M. Liposomes in tissue engineering and regenerative medicine. J R Soc Interface 11, 20140459 (2014).
22. Zylberberg, C. & and Matosevic, S. Bioengineered Liposome-Scaffold Composites As Therapeutic Delivery Systems. Therapeutic Delivery 8, 425-445 (2017).
23. Yang, L. et al. Cell membrane-biomimetic coating via click-mediated liposome fusion for mitigating the foreign-body reaction. Biomaterials 271, 120768 (2021).
24. Kang, M., Lee, C.-S. & Lee, M. Bioactive Scaffolds Integrated with Liposomal or Extracellular Vesicles for Bone Regeneration. Bioengineering 8, 137 (2021).
25. Cheng, R. et al. Advanced liposome-loaded scaffolds for therapeutic and tissue engineering applications. Biomaterials 232, 119706 (2020).
26. Suamte, L. & Babu, P. J. Electrospun based functional scaffolds for biomedical engineering: A review. Nano TransMed 100055 (2024) doi: 10.1016/j.ntm.2024.100055.
27. Phipps, M. C., Clem, W. C., Grunda, J. M., Clines, G. A. & Bellis, S. L. Increasing the pore sizes of bone-mimetic electrospun scaffolds comprised of polycaprolactone, collagen I and hydroxyapatite to enhance cell infiltration. Biomaterials 33, 524-534 (2012).
28. Zamani, F., Amani Tehran, M. & Abbasi, A. Fabrication of PCL nanofibrous scaffold with tuned porosity for neural cell culture. Prog Biomater 10, 151-160 (2021).
29. Liang, D., Hsiao, B. S. & Chu, B. Functional Electrospun Nanofibrous Scaffolds for Biomedical Applications. Adv Drug Deliv Rev 59, 1392-1412 (2007).
30. Damaraju, S. M. et al. Three-dimensional piezoelectric fibrous scaffolds selectively promote mesenchymal stem cell differentiation. Biomaterials 149, 51-62 (2017).
31. Beringer, L. T. et al. An electrospun PVDF-TrFe fiber sensor platform for biological applications. Sensors and Actuators A: Physical 222, 293-300 (2015).
32. Mandal, D., Yoon, S. & Kim, K. J. Origin of Piezoelectricity in an Electrospun Poly(vinylidene fluoride-trifluoroethylene) Nanofiber Web-Based Nanogenerator and Nano-Pressure Sensor. Macromolecular Rapid Communications 32, 831-837 (2011).
33. Persano, L. et al. High performance piezoelectric devices based on aligned arrays of nanofibers of poly(vinylidenefluoride-co-trifluoroethylene). Nat Commun 4, 1633 (2013).
34. Correa, S. et al. Highly Scalable, Closed-Loop Synthesis of Drug-Loaded, Layer-by-Layer Nanoparticles. Advanced Functional Materials 26, 991-1003 (2016).
35. Correa, S., Boehnke, N., Deiss-Yehiely, E. & Hammond, P. T. Solution Conditions Tune and Optimize Loading of Therapeutic Polyelectrolytes into Layer-by-Layer Functionalized Liposomes. ACS Nano 13, 5623-5634 (2019).
36. Augustine, R. et al. Electrospun poly(vinylidene fluoride-trifluoroethylene)/zinc oxide nanocomposite tissue engineering scaffolds with enhanced cell adhesion and blood vessel formation. Nano Res. 10, 3358-3376 (2017).
37. Genchi, G. G. et al. Ultrasound-activated piezoelectric P(VDF-TrFE)/boron nitride nanotube composite films promote differentiation of human SaOS-2 osteoblast-like cells. Nanomedicine: Nanotechnology, Biology and Medicine 14, 2421-2432 (2018).
38. Bar-Cohen, Y. & Zhang, Q. Electroactive Polymer Actuators and Sensors. MRS Bulletin 33, 173-181 (2008).
39. Li, C. et al. Flexible Dome and Bump Shape Piezoelectric Tactile Sensors Using PVDF-TrFE Copolymer. Journal of Microelectromechanical Systems 17, 334-341 (2008).
40. Correa, S. et al. Injectable Nanoparticle-Based Hydrogels Enable the Safe and Effective Deployment of Immunostimulatory CD40 Agonist Antibodies. Advanced Science 9, 2103677 (2022).
41. Decher, G. Fuzzy Nanoassemblies: Toward Layered Polymeric Multicomposites. Science 277, 1232-1237 (1997).
42. Lipton, J., Weng, G.-M., Rohr, J. A., Wang, H. & Taylor, A. D. Layer-by-Layer Assembly of Two-Dimensional Materials: Meticulous Control on the Nanoscale. Matter 2, 1148-1165 (2020).
43. Borges, J. & Mano, J. F. Molecular Interactions Driving the Layer-by-Layer Assembly of Multilayers. Chem. Rev. 114, 8883-8942 (2014).
44. dos Santos, V. L. S. et al. Layer-by-layer assembly: A versatile approach for tailored biomedical films and drug delivery. Journal of Drug Delivery Science and Technology 91, 105243 (2024).
45. Lovinger, A. J. Ferroelectric Polymers. Science 220, 1115-1121 (1983).
46. Lee, S. W., Kim, B.-S., Chen, S., Shao-Horn, Y. & Hammond, P. T. Layer-by-Layer Assembly of All Carbon Nanotube Ultrathin Films for Electrochemical Applications. J. Am. Chem. Soc. 131, 671-679 (2009).
47. Calcutt, R., Vincent, R., Dean, D., Arinzeh, T. L. & Dixit, R. Plant cell adhesion and growth on artificial fibrous scaffolds as an in vitro model for plant development. Science Advances 7, eabj1469 (2021).
48. Li, Y., Liao, C. & Tjong, S. C. Electrospun Polyvinylidene Fluoride-Based Fibrous Scaffolds with Piezoelectric Characteristics for Bone and Neural Tissue Engineering. Nanomaterials (Basel) 9, 952 (2019).
49. Bhadwal, N., Ben Mrad, R. & Behdinan, K. Review of Piezoelectric Properties and Power Output of PVDF and Copolymer-Based Piezoelectric Nanogenerators. Nanomaterials (Basel) 13, 3170 (2023).
50. Chen, J.-X., Li, J.-W., Cheng, C.-C. & Chiu, C.-W. Piezoelectric Property Enhancement of PZT/Poly(vinylidenefluoride-co-trifluoroethylene) Hybrid Films for Flexible Piezoelectric Energy Harvesters. ACS Omega 7, 793-803 (2022).
51. Van Audenaerde, J. R. et al. Novel combination immunotherapy for pancreatic cancer: potent anti-tumor effects with CD40 agonist and interleukin-15 treatment. Clin Transl Immunology 9, e1165 (2020).
52. Sico, G., Montanino, M., Loffredo, F., Borriello, C. & Miscioscia, R. Corona Poling Enabling Gravure Printing of Electroactive Flexible PVDF-TrFE Devices. Materials (Basel) 18, 22 (2024).
53. Zhu, R. & Wang, Z. 9-Piezoelectric one- to two-dimensional nanomaterials for vibration energy harvesting devices. in Emerging 2D Materials and Devices for the Internet of Things (eds Tao, L. & Akinwande, D.) 221-241 (Elsevier, 2020). doi: 10.1016/B978-0-12-818386-1.00009-6.
54. Phillips, J. C. & Van Vechten, J. A. Charge Redistribution and Piezoelectric Constants. Phys. Rev. Lett. 23, 1115-1117 (1969).
55. Sukhorukov, G. B. et al. Stepwise polyelectrolyte assembly on particle surfaces: a novel approach to colloid design. Polymers for Advanced Technologies 9, 759-767 (1998).
56. Caruso, F., Caruso, R. A. & Mohwald, H. Nanoengineering of inorganic and hybrid hollow spheres by colloidal templating. Science 282, 1111-1114 (1998).
57. Zhu, R. et al. Poling-free energy harvesters based on robust self-poled ferroelectric fibers. Nano Energy 50, 97-105 (2018).
58. Yang, X., Shah, J. D. & Wang, H. Nanofiber Enabled Layer-by-Layer Approach Toward Three-Dimensional Tissue Formation. Tissue Engineering Part A 15, 945-956 (2009).
59. Truong, Y. B., Glattauer, V., Briggs, K. L., Zappe, S. & Ramshaw, J. A. M. Collagen-based layer-by-layer coating on electrospun polymer scaffolds. Biomaterials 33, 9198-9204 (2012).
60. Catros, S. et al. Layer-by-Layer Tissue Microfabrication Supports Cell Proliferation In Vitro and In Vivo. Tissue Engineering Part C: Methods 18, 62-70 (2012).
61. Flores-Rojas, G. G. et al. Electrospun Scaffolds for Tissue Engineering: A Review. Macromol 3, 524-553 (2023).
62. Ji, W. et al. Bioactive Electrospun Scaffolds Delivering Growth Factors and Genes for Tissue Engineering Applications. Pharm Res 28, 1259-1272 (2011).
63. Nie, H. & Wang, C.-H. Fabrication and characterization of PLGA/HAp composite scaffolds for delivery of BMP-2 plasmid DNA. J Control Release 120, 111-121 (2007).
64. Nie, H., Ho, M.-L., Wang, C.-K., Wang, C.-H. & Fu, Y.-C. BMP-2 plasmid loaded PLGA/HAp composite scaffolds for treatment of bone defects in nude mice. Biomaterials 30, 892-901 (2009).
65. Sakai, S., Yamada, Y., Yamaguchi, T., Ciach, T. & Kawakami, K. Surface immobilization of poly(ethyleneimine) and plasmid DNA on electrospun poly(L-lactic acid) fibrous mats using a layer-by-layer approach for gene delivery. Journal of Biomedical Materials Research Part A 88A, 281-287 (2009).
66. Zeng, J. et al. Poly(vinyl alcohol) Nanofibers by Electrospinning as a Protein Delivery System and the Retardation of Enzyme Release by Additional Polymer Coatings. Biomacromolecules 6, 1484-1488 (2005).
67. Yang, Y. et al. Structural stability and release profiles of proteins from core-shell poly (DL-lactide) ultrafine fibers prepared by emulsion electrospinning. Journal of Biomedical Materials Research Part A 86A, 374-385 (2008).
68. Li, Y., Jiang, H. & Zhu, K. Encapsulation and controlled release of lysozyme from electrospun poly(ε-caprolactone)/poly(ethylene glycol) non-woven membranes by formation of lysozyme-oleate complexes. J Mater Sci: Mater Med 19, 827-832 (2008).
69. Xiaoqiang, L. et al. Fabrication and properties of core-shell structure P(LLA-CL) nanofibers by coaxial electrospinning. Journal of Applied Polymer Science 111, 1564-1570 (2009).
70. Li, H. et al. Controlled Release of PDGF-bb by Coaxial Electrospun Dextran/Poly(L-lactide-co-&-caprolactone) Fibers with an Ultrafine Core/Shell Structure. Journal of Biomaterials Science, Polymer Edition 21, 803-819 (2010).
71. Liao, I., Chew, SY & and Leong, K. Aligned Core-Shell Nanofibers Delivering Bioactive Proteins. Nanomedicine 1, 465-471 (2006).
72. Suk Choi, J. & Sang Yoo, H. Electrospun Nanofibers Surface-modified with Fluorescent Proteins. Journal of Bioactive and Compatible Polymers 22, 508-524 (2007).
73. Grafahrend, D. et al. Control of protein adsorption on functionalized electrospun fibers. Biotechnology and Bioengineering 101, 609-621 (2008).
74. Li, W. et al. Electrospun nanofibers immobilized with collagen for neural stem cells culture. J Mater Sci: Mater Med 19, 847-854 (2008).
75. Hu, X., Wang, T., Li, F. & Mao, X. Surface modifications of biomaterials in different applied fields. RSC Advances 13, 20495-20511 (2023).
76. Correa, S., Boehnke, N., Deiss-Yehiely, E. & Hammond, P. T. Solution Conditions Tune and Optimize Loading of Therapeutic Polyelectrolytes into Layer-by-Layer Functionalized Liposomes. ACS Nano 13, 5623-5634 (2019).
77. Alavi, M., Karimi, N. & Safaci, M. Application of Various Types of Liposomes in Drug Delivery Systems. Adv Pharm Bull 7, 3-9 (2017).
78. Bozzuto, G. & Molinari, A. Liposomes as nanomedical devices. International Journal of Nanomedicine 10, 975-999 (2015).
79. Correa, S., Grosskopf, A. K., Klich, J. H., Lopez Hernandez, H. & Appel, E. A. Injectable liposome-based supramolecular hydrogels for the programmable release of multiple protein drugs. Matter 5, 1816-1838 (2022).
80. Nsairat, H. et al. Liposomes: structure, composition, types, and clinical applications. Heliyon 8, e09394 (2022).
81. Wolf, H. & Gingell, D. Conformational response of the glycocalyx to ionic strength and interaction with modified glass surfaces: study of live red cells by interferometry. J Cell Sci 63, 101-112 (1983).
82. Bussin, B., MacDuff, M. G. G., Ngo, W. & Chan, W. C. W. Cellular Glycocalyx Affects Nanoparticle Access to Cell Membranes and Uptake. Advanced Materials 37, 2503004 (2025).
83. Tien, J., Terfort, A. & Whitesides, G. M. Microfabrication through Electrostatic Self-Assembly. Langmuir 13, 5349-5355 (1997).
84. Li, R., Zhou, J., Liu, H. & Pei, J. Effect of Polymer Matrix on the Structure and Electric Properties of Piezoelectric Lead Zirconatetitanate/Polymer Composites. Materials 10, 945 (2017).
85. Abdolmaleki, H., Haugen, A. B., Buhl, K. B., Daasbjerg, K. & Agarwala, S. Interfacial Engineering of PVDF-TrFE toward Higher Piezoelectric, Ferroelectric, and Dielectric Performance for Sensing and Energy Harvesting Applications. Advanced Science 10, 2205942 (2023).
86. Wu, S. et al. Aligned Fibrous PVDF-TrFE Scaffolds with Schwann Cells Support Neurite Extension and Myelination In Vitro. J Neural Eng 15, 056010 (2018).
87. Lee, Y.-S., Collins, G. & Livingston Arinzeh, T. Neurite extension of primary neurons on electrospun piezoelectric scaffolds. Acta Biomaterialia 7, 3877-3886 (2011).
88. Gryshkov, O. et al. PVDF and P(VDF-TrFE) Electrospun Scaffolds for Nerve Graft Engineering: A Comparative Study on Piezoelectric and Structural Properties, and In Vitro Biocompatibility. International Journal of Molecular Sciences 22, (2021).
89. Ni, Q. et al. A tug of war between filament treadmilling and myosin induced contractility generates actin rings. eLife 11, e82658 (2022).
90. Maric, S. et al. Time-resolved small-angle neutron scattering as a probe for the dynamics of lipid exchange between human lipoproteins and naturally derived membranes. Sci Rep 9, 7591 (2019).
91. Tenchov, R., Bird, R., Curtze, A. E. & Zhou, Q. Lipid Nanoparticles—From Liposomes to mRNA Vaccine Delivery, a Landscape of Research Diversity and Advancement. ACS Nano 15, 16982-17015 (2021).
92. Gonçalves, A., Machado, R., Gomes, A. C. & Costa, A. da. Nanotechnology Solutions for Controlled Cytokine Delivery: An Applied Perspective. Applied Sciences 10, 7098 (2020).
93. Perera, P.-Y., Lichy, J. H., Waldmann, T. A. & Perera, L. P. The role of interleukin-15 in inflammation and immune responses to infection: implications for its therapeutic use. Microbes and Infection 14, 247-261 (2012).
94. Kanegane, H. & Tosato, G. Activation of Naive and Memory T Cells by Interleukin-15. Blood 88, 230-235 (1996).
95. Interleukin (IL) 15 is a novel cytokine that activates human natural killer cells via components of the IL-2 receptor. J Exp Med 180, 1395-1403 (1994).

What is claimed is:

1. A composition comprising:
a) a piezoelectric polymer scaffold; and
b) a plurality of charged nanoparticles, non-covalently adsorbed onto the piezoelectric polymer scaffold.

2. The composition of claim 1, wherein the piezoelectric polymer scaffold comprises a piezoelectric fluoropolymer scaffold.

3. The composition of claim 1, wherein the piezoelectric polymer scaffold is electrospun.

4. The composition of claim 1, wherein the plurality of charged nanoparticles are electrostatically adsorbed onto the piezoelectric polymer scaffold.

5. The composition of claim 1, wherein when the plurality of charged nanoparticles is fluorescently labeled, and the fluorescence is measured via an inverted confocal fluorescence microscope, the mean fluorescence intensity density per micron of the piezoelectric polymer scaffold is from 20 RFU/μm²-40 RFU/μm².

6. The composition of claim 1, wherein the plurality of charged nanoparticles comprises functionalized polymer latex nanoparticles, layer-by-layer coated nanoparticles, and/or liposomes.

7. The composition of claim 1, wherein the plurality of charged nanoparticles comprise functionalized charged nanoparticles comprising an active agent, wherein the active agent comprises one or more of: a protein, an antibody or fragment thereof, a single-chain variable fragment, a chimeric antigen receptor, or a nucleic acid.

8. The composition of claim 6, wherein one or more of the active agents is encapsulated by a functionalized charged nanoparticle and/or is surface mounted to one or more of the functionalized charged nanoparticles.

9. The composition of claim 8, wherein the active agent is surface mounted via a metal-chelating lipid.

10. The composition of claim 1, wherein the plurality of charged nanoparticles comprises liposomes and an immune modulator, optionally wherein the immune modulator is surface mounted to one or more of the plurality of charged nanoparticles via a metal-chelating lipid.

11. The composition of claim 1, further comprising a plurality of biological cells adhered to the piezoelectric polymer scaffold and/or one or more of the plurality of charged nanoparticles or associated therewith.

12. The composition of claim 1, wherein the composition further comprises a plurality of biological cells, optionally wherein the plurality of biological cells comprises mammalian cells.

13. A method of modulating activity in a tissue comprising placing in the tissue the composition of claim 1, so as to thereby modulate activity of the tissue.

14. A method of improving three dimensionality of a cell culture comprising:
a) providing a composition comprising:
i) a piezoelectric polymer scaffold; and
ii) a plurality of charged nanoparticles, non-covalently adsorbed onto the piezoelectric polymer scaffold;
b) applying the composition into a cell culture environment, thereby improving the three dimensionality of the cell culture.

15. The method of claim 14, wherein the cell culture is an immune cell culture and the nanoparticles comprise one or more immune modulators.

16. A method of producing a composition comprising:
a) providing a piezoelectric polymer scaffold, wherein the piezoelectric polymer scaffold is electrospun;
b) providing a plurality of charged nanoparticles;
c) providing a buffer solution;
d) combining the piezoelectric polymer scaffold, the plurality of charged nanoparticles, and the buffer solution;
e) applying a vacuum to the buffer solution, and repeatedly agitating the buffer solution, so as to thereby non-covalently adsorb the plurality of charged nanoparticles onto the piezoelectric polymer scaffold and produce the composition.

17. The method of claim 16, wherein the piezoelectric polymer scaffold is corona poled.

18. The method of claim 16, wherein the pH of the buffer solution is 5-9.

19. The method of claim 16, wherein the ionic strength of the buffer solution is at least 0.4 M.

20. The method of claim 16, wherein the concentration of the plurality of charged nanoparticles in the buffer solution is at least 0.1 mg/mL.
